(19) United States
(12) Patent Application Publication
(10) Pub. No.: US 2018/0104059 A1
(43) Pub. Date: Apr. 19, 2018

(54) Delivery of Therapy to Living Tissue

(71) Applicant: President and Fellows of Harvard College, Cambridge, MA (US)

(72) Inventors: Ellen T. Roche, Galway (IE); Kevin C. Galloway, Nashville, TM (US); William Whyte, Somerville, MA (US); Conor J. Walsh, Cambridge, MA (US); Hugh O'Neill, Dublin (IE); David J. Mooney, Sudbury, MA (US); Garry P. Duffy, Logstown (IE)

(73) Assignees: President and Fellows of Harvard College, Cambridge, MA (US); Royal College of Surgeons in Ireland, Dublin (IE)

(21) Appl. No.: 15/557,353

(22) PCT Filed: Mar. 11, 2016
(86) PCT No.: PCT/US16/21986

§ 371 Date: Sep. 11, 2017

Related U.S. Application Data
(60) Provisional application No. 62/131,385, filed on Mar. 11, 2015.

(51) Int. Cl. A61F 2/24; A61M 5/142; A61M 37/00; A61N 1/05; A61M 39/02; A61K 35/28; A61P 9/00

(52) U.S. Cl. CPC A61F 2/2481; B33Y 80/00; A61M 37/0015; A61N 1/0587; A61M 39/0247; A61K 35/28; A61P 9/00; A61F 2250/0067; A61M 2037/0023; A61M 2037/0061; A61F 2210/0071; A61M 2039/0258; A61F 2210/0014; A61F 2250/0023; A61F 2240/004; A61M 5/14276

(57) ABSTRACT

Therapy is provided to living tissue by contacting the living tissue with at least one reservoir loaded with cells or a therapeutic composition, wherein the reservoir is in fluid communication with at least one conduit that includes a refilling port. A constituent selected from (a) cells, (b) bioagents from the cells or (c) the therapeutic composition is released from the reservoir to the living tissue. The reservoir is then refilled with (i) cells, (ii) nutrients for cells, or (iii) additional therapeutic composition; and (a) cells, (b) bioagents from the cells or (c) the therapeutic composition continue to be released from the reservoir to the living tissue after the refilling.

### BACKGROUND

Stem cell therapy is a promising candidate for treatment of cardiomyopathies and heart failure. Orlic, et al., first reported cardiac repair (reduction of infarct size, increase in ejection fraction) by transplanting bone marrow cells in mice after myocardial infarction [Orlic, et al., “Mobilized bone marrow cells repair the infarcted heart, improving function and survival,” 98 Proc. Natl. Acad. Sci. U.S.A 10344-49 (2001) and Orlic, et al., “Bone marrow cells regenerate infarcted myocardium”, 410 Nature 701-5 (2001)]; and Strauer, et al., confirmed this achievement weeks later in a human patient [Strauer, et al., “Intracoronary, human autologous stem cell transplantation for myocardial regeneration following myocardial infarction”, 126 Dtsch. Med. Wochenschr. 932-38 (2001)]. The mechanism of action was found not to be from transdifferentiation of cells into cardiomyocytes but from paracrine factors [see M. Gnecchi, et al., “Paracrine mechanisms in adult stem cell signaling and therapy”, 103 Circ. Res. 1204-1219 (2008), and M. Gnecchi, et al., “Evidence supporting paracrine hypothesis for Akt-modified mesenchymal stem cell-mediated cardiac protection and functional improvement”, 20 FASEB J. 661-669 (2006)].

Studies with bone marrow cells were extended to patients with chronic ischemic heart failure [B. E. Strauer, et al., “10 Years of Intracoronary and Intramyocardial Bone Marrow Stem Cell Therapy of the Heart: From the Methodological Origin to Clinical Practice”, 58 J. Am. Coll. Cardiol. 1095-1104 (2011)]; and researchers then began investigating cardiac stem cells for heart failure. For example, results of the SCIPIO phase 1 trial demonstrate that autologous cardiac stem cells can improve systolic function and reduce infarct size in patients with post MI heart failure [G. Heusch, “SCIPIO brings new momentum to cardiac cell therapy”, 378 Lancet 1827-28 (2011)]. The CADUCEUS phase 1 trial used autologous cardiosphere derived cells (CDCs) and showed increases in viable myocardium [R. R. Makkar, et al., “Intracoronary cardiosphere-derived cells for heart regeneration after myocardial infarction (CADUCEUS): a prospective, randomised phase 1 trial”, 379 Lancet 895-904 (2012)]. Both trials warrant expansion to phase 2 studies.

One of the major hurdles to successful clinical translation of cardiac cell therapy, however, is poor cell survival, retention and engraftment in the myocardium—a critical requirement for effective treatment, and a possible explanation for the transient clinical benefit in specific studies. Various factors contribute to this phenomenon and include exposure of cells to ischemia and inflammation, mechanical washout of cells, flushing by the coronary vasculature, leakage from the injection site and anoikis. Solutions to this problem may be found in products that combine cells with agents more adhesive to resident tissue [K. L. Christman, et al., “Injectable fibrin scaffold improves cell transplant survival, reduces infarct expansion, and induces neovasculature formation in ischemic myocardium”, 44 J. Am. Coll. Cardiol. 654-660 (2004)]. Multiple studies have corroborated preliminary findings in our lab that biomaterial delivery vehicles can enhance cellular retention.

Additionally, regenerative therapy for the diseased heart (in the form of cells, macromolecules and small molecules) also has faced multiple hurdles, including poor retention, short biological half-life, adverse side effects from systemic delivery, and the need for multiple administrations.

### SUMMARY

A method for providing therapy to living tissue and a tissue therapy apparatus are described herein, where various embodiments of the apparatus and methods may include some or all of the elements, features and steps described below.

In a method for providing therapy to living tissue, living tissue is contacted with at least one reservoir loaded with cells or a therapeutic composition, wherein the reservoir is in fluid communication with at least one conduit (e.g., a catheter) that includes a refilling port. A constituent selected from (a) cells, (b) bioagents from the cells or (c) the therapeutic composition is released from the reservoir to the living tissue. The reservoir is then refilled with (i) cells, (ii) nutrients for cells, or (iii) additional therapeutic composition; and (a) cells, (b) bioagents from the cells or (c) the therapeutic composition continue to be released from the reservoir to the living tissue after the refilling.

The reservoir can be designed to increase retention at the tissue site and to provide controlled, targeted and replenishable localized release to the tissue. The reservoir can be engineered to provide immunological protection to its biological constituents.

In another method for providing therapy to living tissue, living tissue is contacted with a sleeve through which conduits pass, wherein the conduits each include a first open end in fluid communication with the living tissue, with a biomaterial on the tissue, or with a reservoir containing the biomaterial and including a porous membrane at an interface with the tissue; and at least one of (a) cells, (b) bioagents from the cells and (c) the therapeutic composition is periodically injected from the catheter into contact with the living tissue. In particular embodiments, a catheter is inserted through at least one of the conduits, wherein the injection is performed via the catheter.

A tissue therapy apparatus includes at least one reservoir including a porous wall through which contents of the reservoir can pass; a conduit including a first end and a second end, wherein the second end is in fluid communication with the reservoir; and a refill port mounted at the second end of the conduit. In addition to a refill port, the apparatus can include an extracorporeal or intracorporeal pump and reservoir. The pump reservoir can be filled transcutaneously or worn on a belt like an insulin pump.

Cardiac cell therapy is an emerging therapy that has been limited by poor retention or engraftment of cells in the heart, though the use of a therapeutic layer or sleeve that surrounds the heart and allows replenishable or refillable delivery of therapy to biomaterials, as described herein, can provide for superior cell retention, and superior clinical benefit. The pericardium is a fibrous layer that surrounds the heart. In particular embodiments, the sleeve can serve as a replacement pericardium (made of synthetic or natural biomaterials) that allows sustained and controllable delivery of therapy, or a therapeutic pericardium, which we refer to as a “thericardium.” By combining a biomaterial cell carrier that allows replenishment of cells to the myocardium with a passive restraint layer, the methods and apparatus described herein can potentially promote “reverse remodeling” [as described in M. C. Oz, et al., “Direct cardiac compression devices”, 21 J. Heart Lung Transplant 1049-055 (2002) and in H. R. Levin, et al., “Reversal of Chronic Ventricular Dilation in Patients With End-Stage Cardiomyopathy by Prolonged Mechanical Unloading”, 91 Circulation 2717-720 (1995)] and myocardial restoration. Where a patient has suffered a heart attack, for example, the apparatus can release therapy to restrict the growth of scar tissue on/in the heart. The apparatus can also provide therapeutic benefit as a passive restraint device.

The thericardium system, described herein, offers a number of advantageous features to address the current limitations for the delivery of cells, macromolecules and small molecules to treat cardiac disease. A reservoir in the thericardium can be directly placed on the heart and connected to a subcutaneous port through an implanted conduit or catheter, allowing a localized, targeted therapy to the diseased tissue, without the need for higher systemic doses. This reservoir can house a pre-loaded and refillable biomaterial for sustained delivery of therapy, and a surgical method of implantation in a rat model is introduced that enables repeated replenishment of therapy from a subcutaneous port. As biomaterials have been shown to increase retention in this type of cargo delivery, a biomaterial reservoir can be used. For example, a methacrylated gelatin cryogel can be used, but we foresee this platform system being used with numerous types of biomaterials. In a further refinement, a second rate-limiting membrane can be introduced in the reservoir, thus offering a method to tune the rate of therapy diffusion into tissue, and the size of molecules permitted through the membrane. The delivery of cells is demonstrated using luciferase-expressing mouse mesenchymal stem cells, proteins using fluorescently tagged bovine serum albumin, and small molecules using D-luciferin, an imaging substrate that causes bioluminescence in the presence of the enzyme luciferase and oxygen. In various embodiments the reservoir has a controlled release mechanism for bioagent and/or therapeutic composition release. Other embodiments omit such a mechanism.

### BRIEF DESCRIPTION OF THE DRAWINGS

FIG. 1 illustrates a thermal process for forming the cardiac sleeve 12 of a thericardium.

FIG. 2 shows a single reservoir 34 containing a biomaterial 42 in the form of a dehydrated shape memory alginate gel that can be rehydrated in situ and that can also be seeded with cells in the pocket of the reservoir 34; also shown is a measure, at top, in inches.

FIG. 3 shows a single reservoir 34 containing a cryogel biomaterial 44 with a measure, at top, in inches. The cryogel 42 can be pre-seeded with cells or loaded with therapy.

FIG. 4 shows a thericardium 10 around a heart 14 and subcutaneous port 18 leading to internal channels 16 in the sleeve 12.

FIG. 5 shows a thoracotomy 20 used to place the cardiac sleeve 12 with a catheter 22. A subcutaneous port 18 can be used to refill therapy through the skin via a syringe 24

FIG. 6 plots the radiance (photons/second) as a function of time over 24 hours from the imaging of cells on biomaterial on a rat heart delivered by a thericardium as a therapeutic sleeve anatomically shaped to the heart with a subcutaneous port for a gel (leading to the anatomically shaped sleeve and biomaterial), alone (plot 28), and for a gel with a thericardium (plot 26).

FIGS. 7 and 8 show a cardiac sleeve 12 including a biomaterial liner 19 initially seeded with cells 30, including replenishment reservoirs 34 for the cells refilled via conduits 16 connected with a syringe 34, wherein the biomaterial liner 19 is surrounded by a direct cardiac compression device 32; the exact heart shape can be obtained from MRI/CT scans and reconstructed to fabricate a sleeve 12 for patient specific geometry.

FIG. 9 shows a thericardium sleeve 12 with a network of reservoirs 34 fed by a network of embedded conduits 36 for refilling the reservoirs 34.

FIG. 10 shows another thericardium sleeve 12 with a network of reservoirs 34 with catheters 38 inserted in the conduits 36 that reach the edge and that are used to fill the reservoirs 34.

FIG. 11 shows the thericardium sleeve 12 of FIG. 13 enwrapping a heart 14.

FIG. 12 shows a thericardium sleeve 12 enwrapping a heart 14 and with reservoirs 34 filled by four separate fluid supplies via respective conduits 36 with ports connected to a multi-valved fluid supply junction 46.

FIG. 13 shows a thericardium sleeve 12 formed from a double layer of urethane with conduit 16 and reservoirs 34 formed by grooves and indents in the mold. In this embodiment, the conduit 16 is a common embedded conduit and is used to fill and refill each of the reservoirs 34.

FIG. 14 shows another embodiment of a thericardium sleeve 12, wherein many of the reservoirs 34 contain a biomaterial 42 on which cells can be cultured.

FIGS. 15 and 16 respectively plot the effect of mechanical stimulation on migration 104 and Vascular endothelial growth factor (VEGF) release in comparison with static migration 102.

FIG. 17 shows a cardiac sleeve 12 and direct cardiac compression device 32 for combined mechanical and biological therapy.

FIG. 18 shows a rat-implantation model of a thericardium 10, including a subcutaneous port 18 coupled via a catheter 22 with a thericardium sleeve 12 for the rat's heart, wherein the thericardium sleeve 12 was shaped to the rat's heart casting in a 3-D printed mold in the same geometry as a rat's heart of the same size.

FIG. 19 shows an explanted version of a thericardium 10, wherein a catheter 22 is attached to a sleeve 12 that includes a biomaterial and that is sutured to the epicardial surface of a heart 14 for the ex vivo delivery of biological therapies to sleeve 12 and to the heart 14.

FIG. 20 is a schematic illustration of a rat model 50 with a biomaterial reservoir 34 on the heart 14 and an implanted catheter 22 leading to the reservoir 34 that can be refilled with drugs 52, proteins 54, or cells 30 for the localized, targeted, repeatable delivery of cardiac therapy.

FIGS. 21-23 demonstrate the refill of cell therapy with a thericardium.

FIG. 21 plots the bioluminescence (on a logarithmic scale) as a function of time (days since implantation) for (a) direct delivery of cells to the heart without refill 56 and (b) direct delivery with refill 58.

FIG. 22 plots the area under the curves of FIG. 21 for (a) direct delivery of cells 56 and (b) direct delivery with refill 58.

FIG. 23 is an image of a rat showing the bioluminescence from cell delivery after four days for (a) direct delivery without refill (left) and (b) direct delivery after refill (right).

FIGS. 24-26 demonstrate the refill of direct small molecule delivery with a thericardium.

FIG. 24 is an image of a rat showing the bioluminescence from cell delivery after four days for (a) delivery of imaging agent luciferin (model small molecule) via intraperitoneal (IP) systemic injection (left) and (b) delivery of Luciferin via direct thericardium injection (right).

FIG. 25 plots the bioluminescence (on a logarithmic scale) as a function of time (minutes since injection) for (a) direct delivery of small molecules to the heart 60 and (b) intraperitoneal delivery of small molecules 62.

FIG. 26 plots the area under the curves of FIG. 25 for (a) direct delivery s 60 and (b) intraperitoneal delivery 62.

FIG. 27 plots the fluorescence of fluorescently-tagged bovine serum album (protein) in rats as a function of time (in minutes) post-injection for direct injection 64 and for intraperitoneal delivery 66.

FIG. 28 provides images of the fluorescence from the intraperitoneal protein delivery to a rat (left) and from direct protein delivery to a rat (right).

FIG. 29 shows a refillable thericardium 10 with a therapy reservoir 34, a refill port 70, and a refill catheter 22 for supplying therapy from the refill port 70 to the therapy reservoir 34.

FIG. 30 schematically shows how an encapsulated therapy reservoir 34, including a therapy-laden biomaterial 42 encapsulated in an impermeable membrane 76 with a membrane with tunable porosity 78 in contact with diseased tissue 80; the device also includes a refill catheter 22 for supplying additional therapy to the biomaterial 42.

FIG. 31 provides representative bioluminescent images for an encapsulated thericardium device without refill at 1, 4, 7, 10, and 14 days after implantation.

FIG. 32 provides representative bioluminescent images for an encapsulated thericardium device with refill (on day 4) at 1, 4, 7, 10, and 14 days after implantation.

FIG. 33 plots the bioluminescence (on a logarithmic scale) for each group (without refill 82 and with refill 84) as a function of time (days post-implantation) for the implantations shown in FIGS. 31 and 32.

FIG. 34. plots the areas under the curves for both groups plotted in FIG. 33.

FIG. 35 shows an in vivo pump system, including an infusion pump 88 and a therapy supply 90 for an encapsulated therapy reservoir 34.

FIG. 36 shows an exploded view of an encapsulated therapy reservoir 34 including a thermoplastic urethane pocket 76, a cryogel biomaterial 42 in the thermoplastic urethane pocket 76 with a polycarbonate membrane 78 with modifiable pore size and a bottom layer 86 defining a window for the polycarbonate membrane 78, a refill port 70 connected with the thermoplastic urethane pocket 76 via a catheter 22.

FIG. 37 is a partially exploded view of a bifurcated encapsulated thericardium 10 with a dual port 70 and respective catheters 22 feeding (a) a first reservoir 34′ comprising a first thermoplastic urethane pocket 76′ containing a cryogel 42 and including surface layers including a polycarbonate membrane 78, a thermoplastic urethane window layer 86, and a thermoplastic urethane membrane 92 and (b) a second reservoir 34″ comprising a second thermoplastic urethane pocket 76″.

FIG. 38 shows a dual channel encapsulated thericardium 10 with a dual port 70 and respective catheters 22 feeding (a) a first reservoir 34′ comprising a first thermoplastic urethane pocket 76′ serving as a cell reservoir pocket, containing a sheer thinning biomaterial 42, and including a polycarbonate membrane 78 and (b) a second reservoir 34″ comprising a second thermoplastic urethane pocket 76″.

FIG. 39 is a sectional view of an encapsulated therapy reservoir 34 with therapy encapsulated in a biomaterial 42, wherein pulsatile fluid pressure is injected through catheter 22 into the impermeable thermoplastic urethane pocket 76, and wherein the pressure collapses the pocket in which the hydrogel 42 is contained to release its content through the membrane 78.

FIG. 40 shows a clinical application where a therapy reservoir 34 includes a semipermeable membrane at the epicardial surface, wherein the thericardium sleeve is placed on the border zone of an infarcted heart and connected by catheter 22 to a subcutaneous pot 18 for refill of therapy.

FIG. 41 shows refilling of a subcutaneous port 18 via a needle 40 connected to a catheter 22, wherein the catheter 22 is connected to and supplied with therapy from a remote therapy supply 90.

FIG. 42 shows the myocardial injection of cells 30 through the cardiac sleeve 12.

In the accompanying drawings, like reference characters refer to the same or similar parts throughout the different views; and apostrophes are used to differentiate multiple instances of the same or similar items sharing the same reference numeral. The drawings are not necessarily to scale; instead, emphasis is placed upon illustrating particular principles in the exemplifications discussed below.

### DETAILED DESCRIPTION

The foregoing and other features and advantages of various aspects of the invention(s) will be apparent from the following, more-particular description of various concepts and specific embodiments within the broader bounds of the invention(s). Various aspects of the subject matter introduced above and discussed in greater detail below may be implemented in any of numerous ways, as the subject matter is not limited to any particular manner of implementation. Examples of specific implementations and applications are provided primarily for illustrative purposes.

Unless otherwise herein defined, used or characterized, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially (though not perfectly) pure, as practical and imperfect realities may apply; e.g., the potential presence of at least trace impurities (e.g., at less than 1 or 2%) can be understood as being within the scope of the description; likewise, if a particular shape is referenced, the shape is intended to include imperfect variations from ideal shapes, e.g., due to manufacturing tolerances. Percentages or concentrations expressed herein can represent either by weight or by volume. Processes, procedures and phenomena described below can occur at ambient pressure (e.g., about 50-120 kPa—for example, about 90-110 kPa) and temperature (e.g., −20 to 50° C.—for example, about 10-35° C.) unless otherwise specified.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments.

Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly.

Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms, “includes,” “including,” “comprises” and “comprising,” specify the presence of the stated elements or steps but do not preclude the presence or addition of one or more other elements or steps.

Additionally, the various components identified herein can be provided in an assembled and finished form; or some or all of the components can be packaged together and marketed as a kit with instructions (e.g., in written, video or audio form) for assembly and/or modification by a customer to produce a finished product.

Inspired by the protective nature of the pericardium, a fibrous sac that surrounds the heart, here we present a new system called “thericardium” (therapeutic pericardium), that enables direct, controllable administration of therapy directly to the heart through one or more polymer-based reservoirs capable of controlled release of therapy. The therapy reservoir is implanted on the heart and can be replenished through a catheter connected to an implanted subcutaneous port. This system presents numerous advantages, including convenient, repeated therapy administration, and rapid, cost-effective in vivo imaging for quantification of targeted therapy. The thericardium can reduce or eliminate scarring and restore full cardiac function post-myocardial infarction and can attenuate or eliminate the cascade of events that lead to heart failure and prevent ischemic cardiomyopathy. As a research model, this system may elucidate new insights into regenerative cardiac therapy and advance experimental therapies along the clinical translational path. Overall, this work has practical implications in enabling experiments that were previously prohibited by cost, invasiveness and quantification challenges.

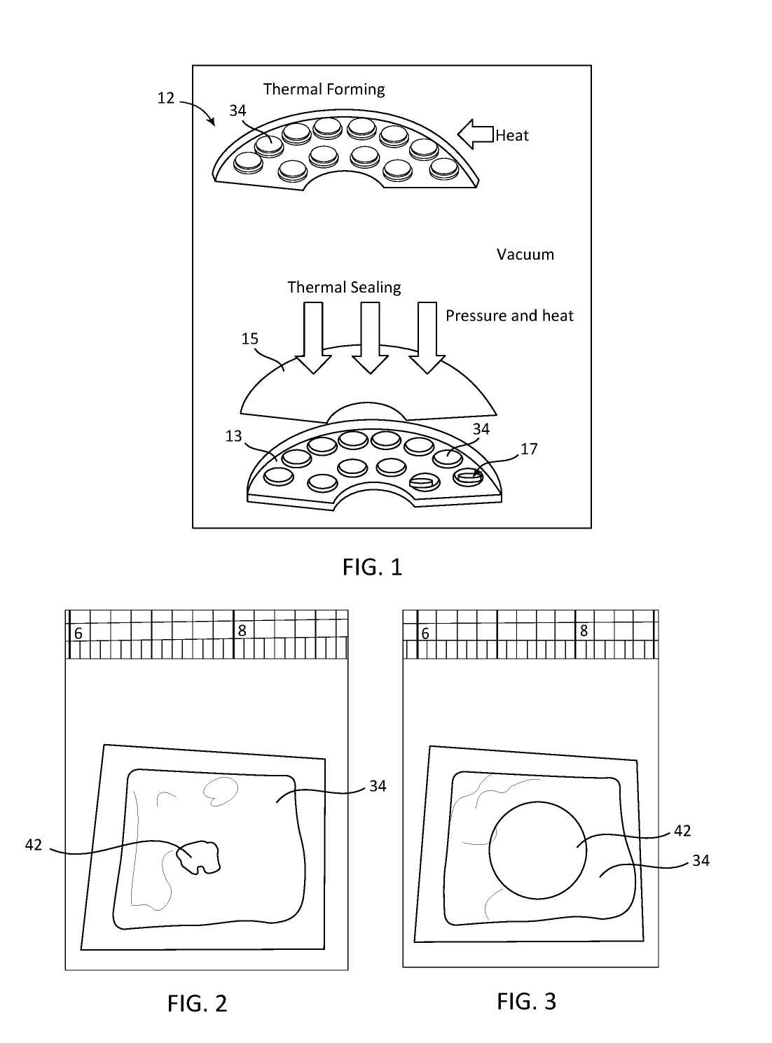

In the embodiment of FIG. 1, a plurality of reservoirs 34 fed by respective feed conduits are embedded in a cardiac sleeve 12 made from a double layer of thermoplastic urethane. One urethane layer 13 that forms the sleeve 12 is thermally formed using a mold to produce the reservoirs 34 and channels inside it. A second layer of urethane 15 is sealed to the first layer 13 to encapsulate the reservoirs 34. The second layer of urethane 15 covering the reservoirs 34 is perforated (e.g., with a laser to provide holes with a diameter of 10-50 μm) to provide it with a porosity (thereby functioning as a porous membrane) that allows the therapy 17 to diffuse through the membrane 15 and out of the reservoirs 34 into contact with the heart (or other tissue). Alternatively, a membrane 15 with a known pore size (e.g., from filters) can be used or manufactured. A thermoplastic polycarbonate membrane 15 with tuneable pore size can also be used for the controlled release of therapy 17. A small pore size can also be used to provide immunological protection to the cells inside the reservoirs 34.

The therapy in the reservoirs 34 and released by the reservoirs 34 can include, e.g., cells, a bioagent produced by cells and that acts on and affects other cells—for example, a paracrine-acting agent, a growth factor, such as a bone morphogenetic protein (BMP) or vascular endothelial growth factor (VEGF); a chemotherapeutic agent; an immunosuppressant; culture media, conditioned media; small molecules that help regulate a biological process; an anti-rejection drug; an anti-arrythmic drug; anti-anginal drug; a cardioprotective drug for use, e.g., during chemotherapy; hormones, dopamine, levodopa, antibiotics, anti-inflammatory drugs, anti-thyroid pharmacotherapy, anti-microbial drugs, and lidocaine; or macromolecules or small molecules.

In particular embodiments, the reservoirs 34 do not all contain the same contents. Rather, different types of therapy are contained in different reservoirs 34, thereby allowing for selective actuation, as described below, of particular reservoirs 34 to deliver a particular therapy at one time and for later selective actuation of another set of reservoirs 34 to deliver a different therapy at a subsequent time. In particular embodiments, varying pressure can be used for a tunable release from respective reservoirs 34. Different reservoirs can be fabricated with different thicknesses or other features that provide for a graded release among the reservoirs 34 in response to pressure. In additional embodiments, valves can be incorporated into the sleeve 12 to provide for on/off release from respective reservoirs 34. A once off burst release can also be used in the reservoirs 34. The reservoirs 34 can also contain biosensors that detect levels of disease-responsive biomarkers (e.g., troponins and matrix metalloproteinases) or that detect the mechanical function of the heart.

For wound healing, the apparatus can include a topical reservoir, such as a transdermal patch or a micro needle patch, attached to a refillable reservoir 34 containing (a) cells, (b) bioagents from the cells or (c) other therapeutic composition (e.g., antibiotic). In this embodiment, the reservoir 34 can be incorporated into a bandage or dressing and contacted with, e.g., wounded or otherwise damaged skin tissue on an external surface of the body.

In particular embodiments, as shown in FIGS. 2 and 3, a single reservoir 34 can be used and placed in contact with the heart 14. In these embodiments, the reservoir 34 contains a biomaterial 42in the form of a cryogel comprising a dehydrated shape memory alginate gel that can be rehydrated in situ in the pocket of the reservoir 34 and that can be pre-seeded or simultaneously loaded with cells or with therapy.

Where the reservoirs 34 contain biomaterials and cells, the pores can be made very small to restrict passage of cells or biomaterial there through while still being large enough to permit passage of growth factors produced by the cells. In a particular embodiment, the pores of the membrane 15 can have a diameter of about 0.4 μm. A suitable product is thermoplastic urethane commercially available from American Polyfilm, Inc., of Branford, Conn., USA, which can be laser-cut with pores of desired size; alternatively, a polycarbonate porous membrane can be used. In particular embodiments, the reservoir(s) 34 and sleeve 12 (if used) can be formed of a biodegradeable material so that the apparatus can biodegrade to eventually substantially eliminate it from the body, thereby removing the need for a second surgery to remove the apparatus after its use. In additional embodiments, the reservoir(s) 34 or sleeve 12 can include structures/compositions that enable it or its features to show up under x-ray imaging; for example, the device can have radiopaque channels for visibility under x-ray. In further embodiments, the reservoir(s) 34 and/or sleeve 12 are formed of materials that will not interfere with imaging for diagnostic purposes (e.g., magnetic resonance imaging) to assess how the heart is healing over time.

Biomaterials on which cells can be grown, such as injectable biomaterials and thermoresponsive biomaterials, can be fabricated and formed into the reservoir(s) 34. Here, cryogels (i.e., gels that are frozen to produce a porous structure) were formed, though other biomaterials can be substituted for the cryogels. Though this embodiment employs a sleeve 12, one or more reservoirs 34 can be implanted and secured as pockets or patches or otherwise placed in contact with the tissue without being embedded in a sleeve 12. In particular embodiments, reservoirs 34 can be provided with surface properties (e.g., micro-patterning) or adhesive to adhere it to tissue and to remain in contact with the tissue without changing position. Other ways to attach the reservoirs 12 to the heart include the use of functionalized gel, suction (e.g., micro-suckers), sutures, mesh, etc. The adhesive material properties can be such that the adhesive can be elastic when cured so that it can maintain adhesion given movement and deformation of tissue. In addition, the adhesive can be located in a pattern (i.e., not covering the full surface) to aid with adhesion in a dynamic context. In addition, micro-needle technology can be combined with the thericardium to provide an interface to the tissue and enabling injection/infusion of therapy directly into the tissue.

Microneedle technology is a technique for delivery of small molecules and biologics, whereby micron-scale hollow needles are fabricated (borrowing techniques from the micro-electronics industry) in patches. Microneedles can be used for transdermal or trans-tissue delivery of biologics and are promising micro-fabricated devices for minimally invasive drug delivery applications. Microneedles are high performance conduits, through which drug solutions may pass into the body and are designed to be as small as possible. Microneedles are also designed to be extremely sharp, with submicron tip radii, allowing the needles to be effectively inserted into the skin. Microneedles offer an attractive way for advanced drug delivery systems by mechanically penetrating the skin and injecting drug just under the stratum corneum where it is rapidly absorbed by the capillary bed into the bloodstream.

In various embodiments, the sleeve 12 and/or reservoir(s) 34 can be implanted on a donor organ before organ transplant. In other embodiments, the sleeve 12 and/or reservoir(s) 34 can be implanted concurrent with other surgery or when implanting a mechanical device in the body. In still other embodiments, the sleeve 12 and/or reservoir(s) 34 can be delivered (e.g., pre-loaded) in a folded or rolled-up configuration through a catheter; for example, a balloon catheter can deliver a sleeve 12 or reservoir 34; or a mechanical delivery catheter can unfold a sleeve 12 or reservoir 34 using a shape memory alloy or polymer. In additional embodiments, the sleeve 12 or reservoir 34 can be delivered via a robotic delivery system, such as the HeartLander robot from The Robotics Institute at Carnegie Mellon University. In other embodiments, the sleeve 12, itself, can be robotic and can move to the desired tissue site using sensing or imaging modalities.

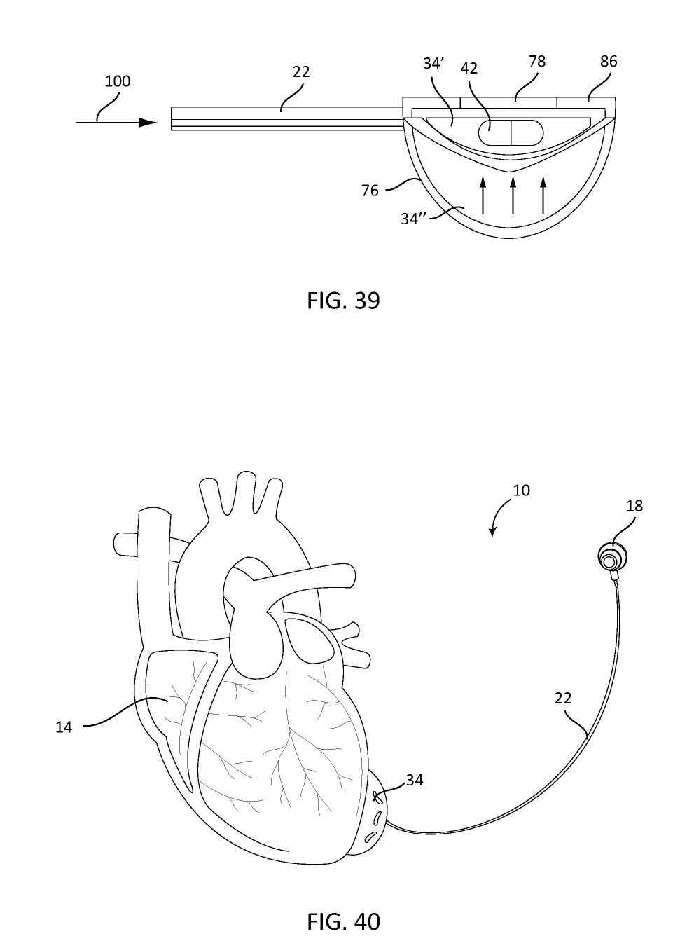

In additional embodiments (as shown in FIG. 39), an actuator (e.g., an electromechanical pump, a soft robotic actuator, a pneumatic or hydraulic pump, a transcutaneous ultrasound device, a magnet, etc.) can be positioned in or against or coupled with the reservoir(s) 34 and can be configured to provide a pulsing motion to discharge contents of the reservoir(s) 34 to the tissue when the actuator is actuated. Cyclical actuation or pressurization of the reservoir 34 can enhance the therapeutic benefit, itself potentially increasing regeneration potential.

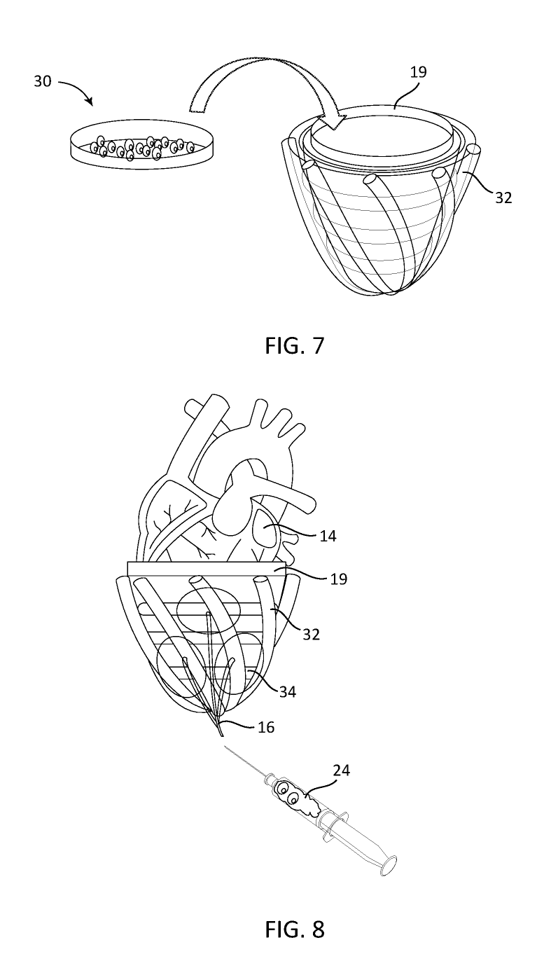

Seeding of cells on cryogels was demonstrated through the channels 16 in a sleeve 12, as illustrated in FIG. 8. Additionally, targeted replenishment (to one or two reservoirs 34 only), global replenishment (to all reservoirs or a subset of reservoirs 34) was demonstrated with dye for visualization and with contrast under x-ray imaging on a porcine heart on the bench and in a live animal. Finally, replenishment of the reservoirs 34 was achieved by tracking a steerable catheter along the channels 16 to refill targeted reservoirs 34 with cells in this example. Alternatively, a steerable catheter with a suction tip can also be used to evacuate the thericardium 10. In additional embodiments, this catheter can have a cold tip to reverse gelate a gel. In still additional embodiments, a degrading enzyme can be injected into the gel half an hour before; and actuation can be used to mix, followed by use of a suction catheter or direct suction applied to the implanted conduit. The reservoirs 34 can be replenished many times—e.g., every 12 hours, every 4 days, etc. The catheter can also have a cold/warm tip to gelate/degrade the gel and/or can have a light-emitting catheter to gelate a functionalized biomaterial 42 for delivery or to degrade a functionalized biomaterial 42 for removal. In additional embodiments, an imaging agent can be injected to visualize the infarcted area/border zone, or a biosensor can be injected to map out the infarcted area.

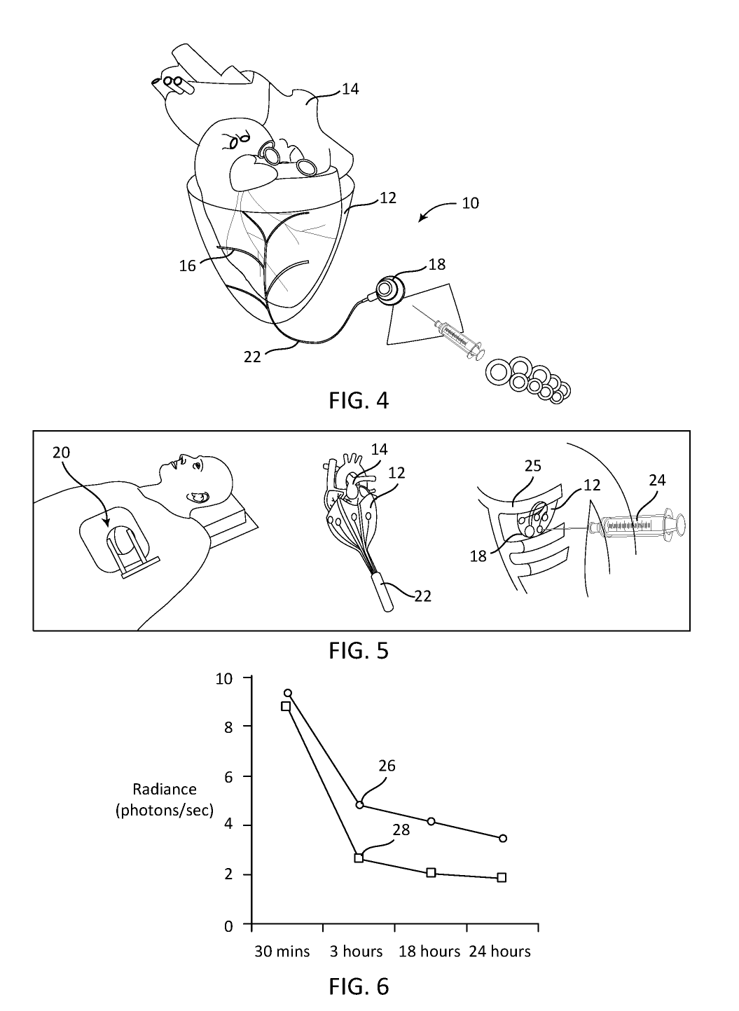

The thericardium 10 can be implanted through a small incision in the ribcage 25 called a thoracotomy, and refilling of therapy (e.g., cells, nutrients for cells, or pharmaceuticals) can be performed through a subcutaneous port 18 (with a self-sealing rubber septum) and conduit 22. In other embodiments, the thericardium 10 can be sufficiently small so that it can be delivered through a catheter, and have a design such that it can expand into a larger shape inside the body. As shown in FIGS. 4 and 5, use of the port 18 provides a minimally invasive way to allow repeated administration of therapy to targeted locations on the heart 14 and can replace current clinical practice where cells are injected directly to the epicardium during a surgical procedure or where cells are injected via a catheter or via an implanted pump and reservoir to the endocardium (or internal wall) of the heart.

### Preliminary Results:

We set up an animal model, where a miniaturized thericardium 10 containing a biomaterial was placed on a rat heart after a myocardial infarction, with the radiance from bioluminescence for a gel and thericardium 26 and for a gel alone 28 plotted in FIG. 6. By connecting the thericardium sleeve 12 to a conduit 22 in the form of a catheter tunneled to a subcutaneous port 18 (embedded between the shoulder blades of the small animal), the system allows minimally invasive replenishment of therapy to the heart 14. Preliminary results show that the procedure is feasible, and refills of therapy can be supplied to the heart. The model can also be used for the directed targeted injection of imaging substrates (for example, luciferin or fluoresceins) directly to the heart to allow fluorescent or bioluminescent imaging of therapy. Fluorescence can be used to track molecules labeled with a fluorescent group, or bioluminescence can be used to track molecules; and the injected imaging substrate can be used to visualize the lighted cells or molecules.

### Combination Mechanical and Biological Therapy:

A recent study has shown that ventricular reloading can induce cardiomyocyte proliferation. D. C. Canseco, et al., Human Ventricular Unloading Induces Cardiomyocyte Proliferation, 65 J. Am. Coll. Cardiol. 892-900 (2015). The authors hypothesized that an increase in mitochondrial content in response to mechanical load causes activation of DNA damage response (DDR) and permanent cell cycle arrest of cardiomyocytes. This impairs the ability of the heart to regenerate. The authors showed that post-LVAD (left ventricular assist device) hearts (after “unloading” of the ventricle) showed a decrease in mitochondrial content and cardiomyocyte size compared with pre-LVAD hearts. If this is the case, the administration of regenerative therapy while the heart is being unloaded should have a better chance of success compared to administration to a heart that is trying to compensate for a volume or pressure overload. As such, there are numerous ongoing trials combining cell therapy with traditional mechanical assist devices. A multimodal combination of cells with mechanical assist devices (passive or active) represents a particularly attractive therapeutic strategy. This approach confers the potential for mechanical devices to act on co-delivered cells, as well as to exert efficacy to the heart. Co-delivery in a biomaterial carrier can ensure that cells are kept in close proximity to the mechanical device for the duration of therapy to enhance synergistic interaction.

In an interesting acellular hybrid therapy approach, Kubota, et al., in “Impact of cardiac support device combined with slow-release prostacyclin agonist in a canine ischemic cardiomyopathy model”, 147 J. Thorac. Cardiovasc. Surg, 1081-1087 (2014), employed an atelocollagen sheet/polyglycolic acid ventricular restraint device (VRD) alone, small molecule PGI2 agonist ONO1301 on an atelocollagen sheet alone, or a multimodal ONO1301-doped VRD in a canine model of myocardial infarction. At 8-weeks post infarction, hearts treated with the multimodal VRD, demonstrated the greatest increase in left ventricle ejection fraction (LVEF) and the greatest reduction in left ventricular wall stress and ventricular remodeling. All hearts treated with ONO1301 (either alone or in combination with a VRD) demonstrated an increase in myocardial vascularization and upregulation of hepatocyte growth factor (HGF), vascular endothelial growth factor (VEGF) and stromal cell-derived factor 1 (SDF-1) in the myocardium. In a similar hybrid approach with cells, Shafy, et al., in “Development of cardiac support bioprostheses for ventricular restoration and myocardial regeneration”, 43 Eur. J. Cardiothorac. Surg. 1211-1209 (2013), showed that the combination of adipose-derived stem cells (injected into the infarct and seeded in a collagen matrix) with a polyester Corcap VRD device resulted in significant improvements in ejection fraction and systolic and diastolic function in a sheep infarct mode. This semi-degradable ventricular bioprosthesis approach is an example of biomaterial-mediated cell therapy combined with a constraint device. The CELLWAVE study [B. Assmus, et al., “Effect of shock wave-facilitated intracoronary cell therapy on LVEF in patients with chronic heart failure: the CELLWAVE randomized clinical trial”, 309 JAMA 1622-1631 (2013)] addressed delivery of BM-MSCs combined with a pretreatment of low energy cardiac shockwave to improve honing of cells and expression of SDF-1 and VEGF. The combination of a shock wave with cells resulted in an increase in an ejection fraction of 3.2%. In Chachques, et al., “Development of bioartificial myocardium using stem cells and nanobiotechnology templates”, 2011 Cardiol. Res. Pract. 806795 (2010), nanobiomaterials with elastomeric membranes were bioengineered to acquire a controlled drug release patch, which they can tailor for local cell attraction and cell differentiation. A phase 1 clinical trial began in August 2009 to test a patch called ANGINERA (from Theregen, Inc., of San Francisco, Calif.) containing cells secreting factors to stimulate growth of other cells by paracrine signaling. The study groups consist of coronary artery bypass graft (CABG) patients and end-stage heart failure patients with an LVAD device.

In the past few years, the idea of combining mechanical support and cellular therapy synergistically has emerged as a realistic alternative to heart transplantation. This contemporary holistic, hybrid approach for end-stage ischemic heart failure may address the issue of scarce donor hearts for transplantation. The success on combining the therapies relies on refining them individually and maximizing their possible combinatorial efficacy.

In one configuration, a sleeve 12 in the form of a layer at the heart/device interface is provided through which therapy can be delivered. This interface layer can be molded out of a biomaterial (as shown in FIG. 7) or can be thermally formed. A number of inbuilt conduits 16 and reservoirs 34 attached to a subcutaneous or external port allow delivery of therapy to this interface (FIG. 8). This configuration allows for replenishment of therapy in a minimally invasive fashion.

To achieve this vision, a bioreactor was developed to determine the effect of cyclical actuation [similar to that provided by the direct cardiac compression (DCC) device] on cells and biomaterials, as described below. Then, as described below, a manufacturing process was developed for a larger scale version of the thericardium 10. The thericardium 10 includes a sleeve 12 that can be used with the DCC device and that includes multiple reservoirs 34 and connecting channels 16 (as shown in FIG. 8) and multiple catheters that can be refilled minimally invasively. Additionally, experiments are described, below, using the DCC device as a bioreactor, examining cell migration through a rate-limiting membrane from the thericardium reservoirs 34, and measuring the growth factor profile released from cells that are dynamically actuated compared to static cells. Further, in vivo use of this scaled-up thericardium is described in a porcine model where dye or x-ray contrast was used to represent therapy being delivered to the heart. Finally, a preliminary in vivo study is described, where the thericardium and the DCC are used in combination in a porcine model.

To understand how transplanted cells in biomaterial reservoirs 34 may respond to cyclical actuation from a direct cardiac compression device 32, a bioreactor was developed that could be used to examine this effect in vitro. A control system was constructed in a sealed chamber that can be placed in the incubator to provide cyclical pneumatic actuation to a dynamic bioreactor. The control system included four solenoid valves (VQ110U-5M from SMC Corp. of Noblesville, Ind., USA), a microcontroller (AA-021205 Arduino Mega, Arduino), and a miniaturized diaphragm pump (D737-23-01 from Parker Hannifin Corp. of Cleveland, Ohio, USA). A simplified bioreactor was made by taking sections of latex tubing and placing a small pneumatic actuator around the tube sections. A porous pocket into which a cell-laden biomaterial could be placed was positioned at the tube/actuator interface. The actuators were connected to the valve outlets in the control system, and each tube/actuator assembly was placed in a 6-well plate. Media (DMEM from Sigma-Aldrich of St. Louis, Mo., USA) was filled into each well plate. The entire assembly was placed in the incubator and cultured for 72 hours. A static group was compared to a dynamic group (actuated at 1 Hz for 72 hours) for a gelatin methacryloyl (GelMA) and an injectable alginate biomaterial (manufactured as previously described and seeded/encapsulated with 1×10⁶ mouse mesenchymal stem cells). The pockets were made from two layers of thermoplastic urethane (HTM6001, 0.006 inch thick, American Polyfilm, Inc.) that was laser cut to have 500 um pores, spaced by 1 mm in each direction. The two layers were sealed using a heat sealer and employing Teflon tape (from Saint-Gobain S. A. of Courbevoie, France) to selectively mask an area with a 2-cm opening on one side of the pocket for biomaterial insertion and media perfusion.

Preliminary results showed that cell viability (measured by live/dead staining at 72 hours) can be improved with dynamic actuation compared to static culture in the alginate group, but not in the GelMA group. The gelMA gel withstood the actuation and maintained its porous structure when imaged by scanning electron microscopy (SEM).

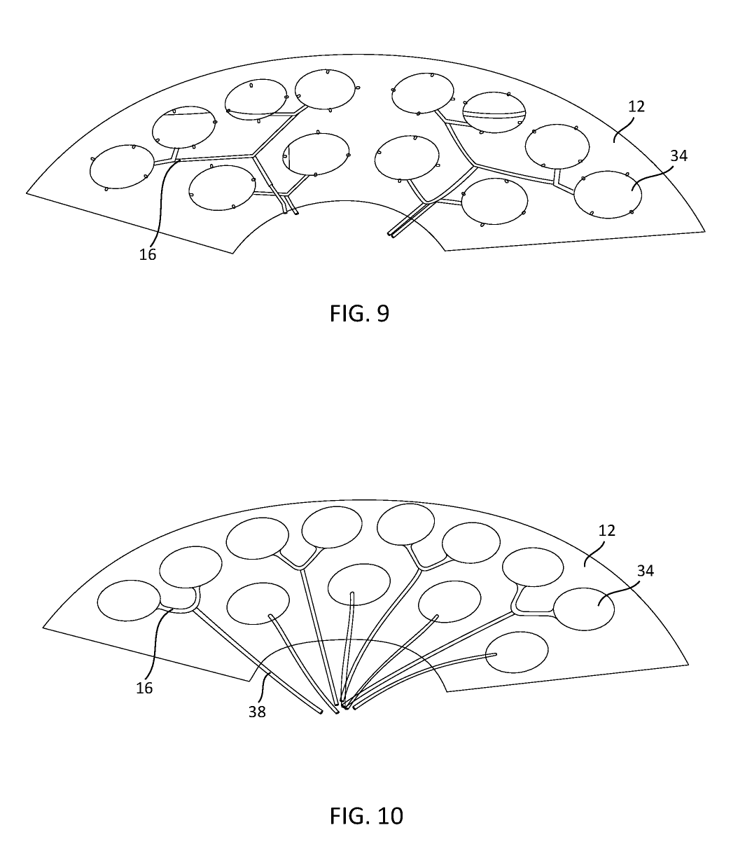

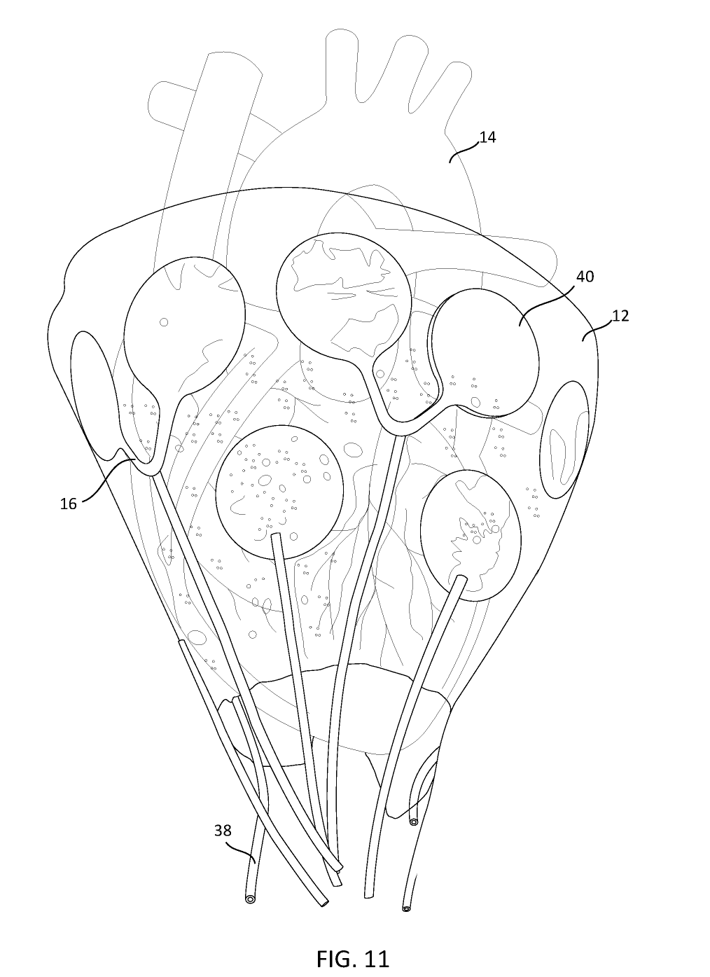

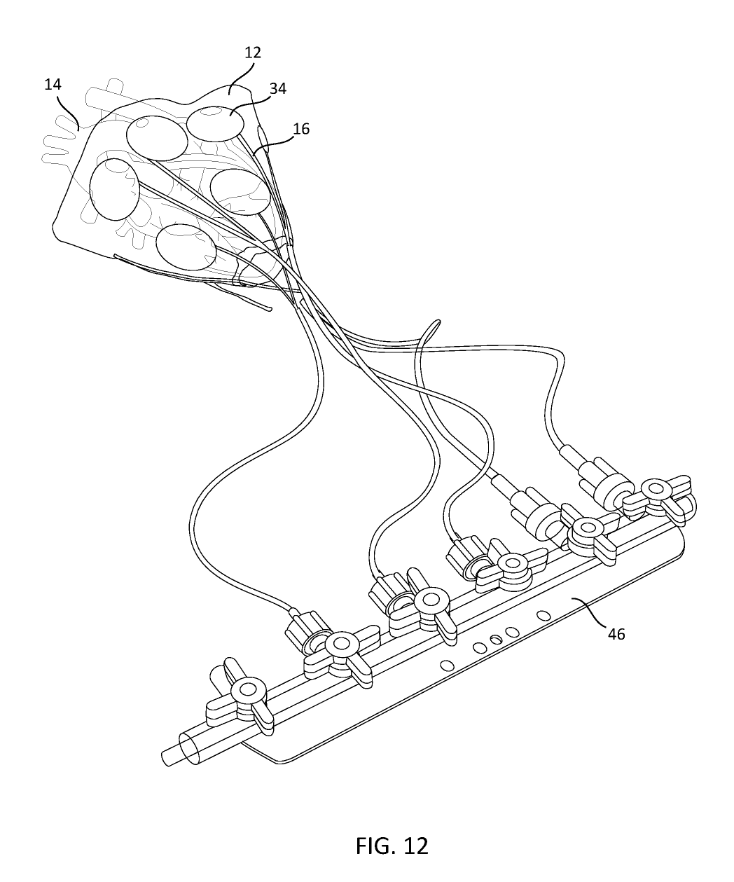

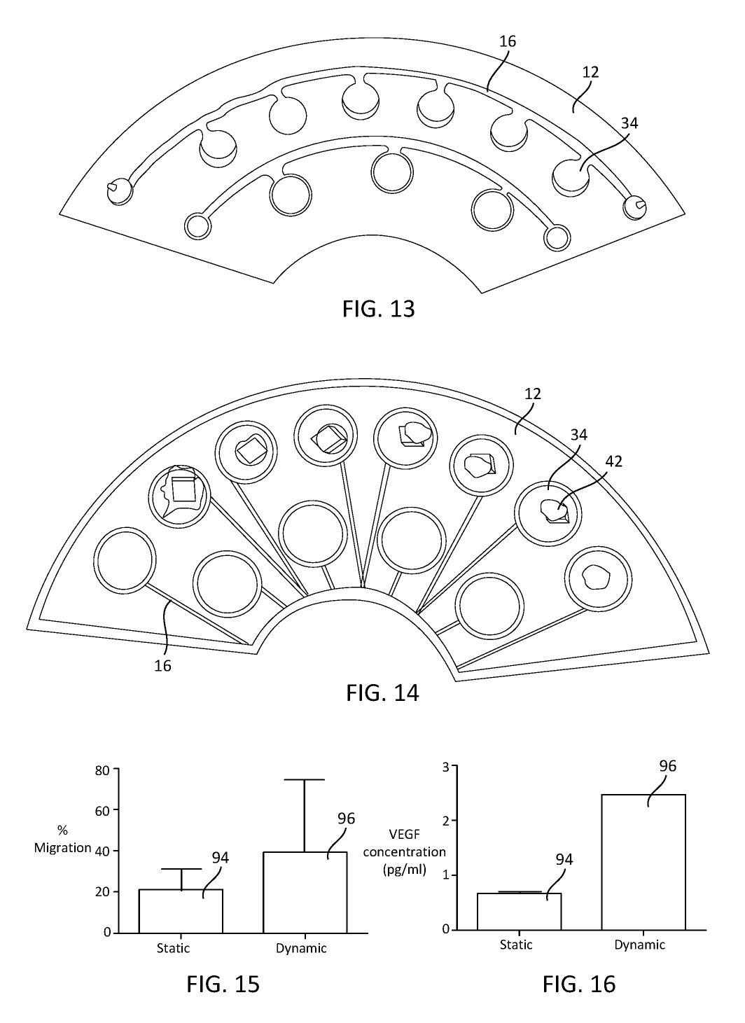

Next, the concept of the thericardium 10 was scaled up so that it could be used in combination with an existing DCC device. To realize this, a manufacturing process was developed to form reservoirs 34 for biomaterials that were connected by channels 16 using sheets of thermoplastic urethane 13 and 15, heat forming and heat sealing, as shown in FIG. 1. A first sheet of thermoplastic urethane 13 (HTM6001, 0.006-inch thick, from American Polyfilm, Inc., of Branford, Conn., USA) is thermally formed (using an EZFORM SY 1917 vacuum forming machine from Centroform of Glendale, Calif., USA) over a 3-D printed mold (using an OBJET CONNEX 500 printer from Stratasys of Edison Prairie, Minn., USA) so that reservoirs 34 and channels 16 (seen in FIGS. 4 and 8-14) are imprinted into it. The geometry of the reservoirs 34 and channels 16 can be modified depending on therapy. The flat pattern was laser cut (using a VERSALASER laser from LST Group of Punchbowl, NSW, Australia) to match the DCC device. Then, a second porous sheet of thermoplastic urethane 15 (HTM 8001-M polyether film 0.003 inch thick, Advanced Polymers, Inc.) is heat sealed to the first layer 13 (heat transfer machine QXAi, Powerpress), after placing lyophilized biomaterials 17 in the reservoirs 34. This forms the rate-limiting layer, and laser cutting small pores (using the VERSALASER laser) can tune the porosity of this layer 15. The entire layer 15 is placed under UV light for decontamination, and the gels can then be rehydrated and seeded simultaneously with cell suspension by injecting through the formed channels. A design feature of this prototype is that reservoirs 34 can be selectively or globally refilled, enabling targeted or selective replenishment.

As the thericardium 10 was now scaled up to the size of the DCC device, the increased size enabled the use of the device as a bioreactor. A double layered reservoir 34 was constructed using the fabrication process previously described with an additional non-porous layer sealed on top. Each reservoir 34 was separated by a 0.003-inch thermoplastic urethane layer (with 500-um laser-cut holes spaced 1 mm apart in each direction). The assembly is shown in FIG. 14. On one side of this membrane were placed squares of Gelfoam absorbable sponge (SKU: 925-4118 from Ace Surgical Supply Co. of Brockton, Mass., USA) to act as a tissue simulant and to allow assessment of migration through the porous membrane. On the other side were placed GelMA cryogels 42, as previously described. The GelMA cryogels 42 were seeded with 1×10⁶ cells (mouse mesenchymal stem cells) in 1 ml of media, and the structure was placed around a silicone heart model underneath the DCC device. The assembly was placed in an incubator; and the airlines were directed out a sealed rubber bung in the back of the incubator for attachment to the pneumatic control box. Media was refilled in the reservoirs 34 every day. The rig was actuated at 1 Hz (with a 200-ms actuation period of the actuators at 10 pounds/in²) for 72 hours.

Some interesting preliminary results were obtained from the experiment, and are shown in FIGS. 15 and 16. Cell migration across the porous membrane, as measured by live-dead staining of the Gelfoam tissue simulant increased with dynamic actuation (plot 96) compared with static migration (plot 94). Also, the vascular endothelial growth factor (VEGF) released from the dynamically actuated cells (using the Mouse VEGF Quantikine ELISA Kit from R&D Systems of Minneapolis, Minn., USA) was higher than the concentration for the static condition.

The thericardium 10 was tested for functionality in a Yorkshire swine model (n=3, 60-70-kg female swine). The sleeve 12 was placed around the heart 14 and dye was used to visualize filling of the channels 16. Contrast was added to the dye, and the procedure was repeated under fluoroscopy to show x-ray filling of the reservoirs 34. The thericardium 10 was refilled via direct injection using a microcatheter that is tracked through the channels 16 to the reservoir 34 to fill one isolated reservoir 34 (blue dye for visualization of filling). Additionally, a fluorescently tagged suspension of alginate beads (50-μm beads tagged with alexafluor −750) was delivered through the thericardium 10, and the heart was imaged using the IVIS Xenogen 5000 imaging system to assess for fluorescence on the tissue. A layer of “tough gel” hydrogel [described in J-Y Sun, et al., “Highly stretchable and tough hydrogels,” 489 Nature 133-36 (September 2012) and comprising 90% water yet stretching without breaking to more than 20 times its original length and recoiling like rubber] was manufactured and placed at the heart/device interface to act as a secondary material reservoir, with the intent of reducing friction between the heart 14 and the thericardium 10 once the DCC device 32 was placed over it and actuated. Finally, a thericardium 10 with incorporated gelfoam was used to explore sustained delivery of drug (in this case epinephrine) to the epicardium of the heart 14. Preliminary in vivo testing showed that the device conformed to the heart well and could be easily attached. Replenishment of the reservoirs 34 with direct injection or catheter injection was possible, and post-trial imaging showed that the therapy was delivered to the myocardium.

Finally, in a preliminary feasibility study, the thericardium 10 and the DCC device 32 were combined on a live porcine model (Yorkshire swine, 60 kg) to evaluate refilling of therapy during active assistance. Refilling was possible and was visualized under x-ray with use of contrast.

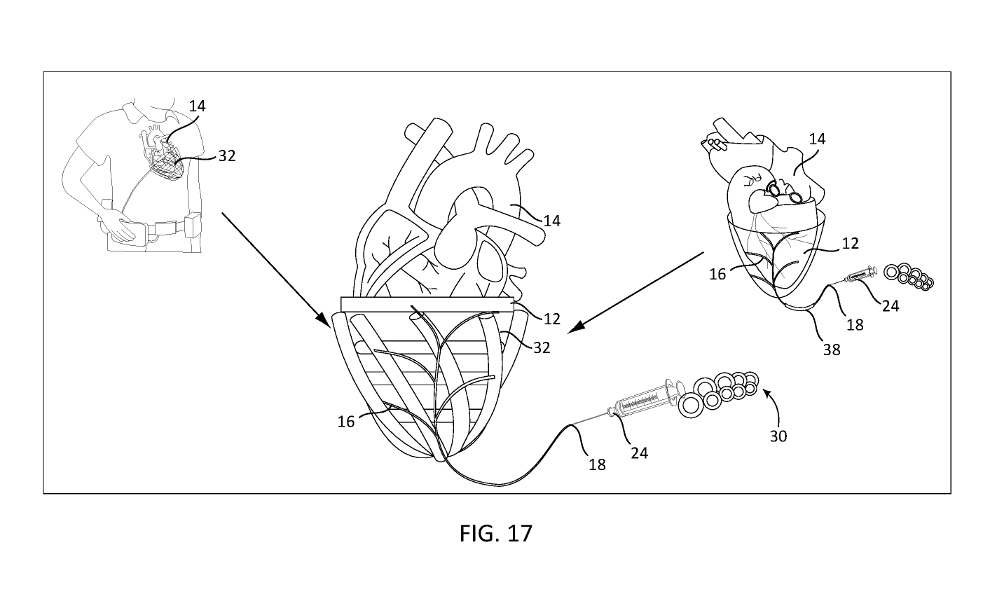

A vision for translation of this combined therapy is two-fold—the thericardium technology can be used to deliver biological therapy with active assist or while it is acting as an adjustable passive restraint device. In a first scenario, a patient receives the thericardium 10 with the DCC device 32, as shown in FIG. 17. Both devices are placed on the heart 14 via a sternotomy. The therapy 30 is refilled through a subcutaneous port. In a second scenario, a patient is treated with the thericardium 10 alone, as shown in FIG. 4, and so receives biological therapy in combination with the mechanical advantage of passive restraint from the sleeve 12 of the thericardium 10. The device is delivered through a mini-thoracotomy. Refill of therapy and adjustment of quantitative restraint are enabled by injecting fluid through the subcutaneous port 28. A pressure sensor enables real-time readout of how much passive restraint is being provided.

### Macromolecule and Small Molecule Therapies:

Both macromolecule and small molecule therapies suffer some similar limitations as cell therapies (i.e., low concentrations at the desired site due to untargeted delivery and a short biological half-life). Delivery of macromolecules represent a promising therapeutic deliverable for the treatment of ischemic cardiomyopathy. The increased accessibility to these bioagents and the advances in chemical modifications to enhance protein half-life in vivo and minimize immunogenicity offer a broad range of new therapeutic modalities. Modified peptides and proteins can enable cardiac repair through activation of endogenous cardiac progenitor cells present at the injury site, the induction of cardiomyocyte proliferation, and the recruitment of progenitor cells to damaged myocardium or cells able to trigger neovascularization. Studies have been conducted with vascular endothelial growth factor (VEGF), stromal cell derived factor (SDF-1), hepatocyte growth factor (HGF), nueregulin (NRG-1), and insulin-like growth factor (IGF-1). The encapsulation of proteins in carrier gels provides a controlled release and enhances retention in the target area. In parallel, advances in synthetic chemistry mean that a library of small molecules can be screened in a biological system to determine novel drug targets and to elucidate previously unknown signaling systems implicated in myocardial disease. Structure activity relationship data can permit and guide molecular amendments to enhance specificity, stability and efficacy. Examples of these molecules include prostaglandin E2 (PGE2), ONO1301, pyrvinium pamoate (PP), or diprotin. A common theme underpinning studies with delivery of these small molecules is the necessity for redelivery or sustained delivery of drugs. It is feasible to conclude that the increased bioavailability of these agents at a pathological site within a suitable therapeutic window, as can be afforded by the thericardium 10, may lead to a new option in the treatment of cardiovascular disease.

### Exemplification

Simplified Refillable Thericardium in an Animal Model:

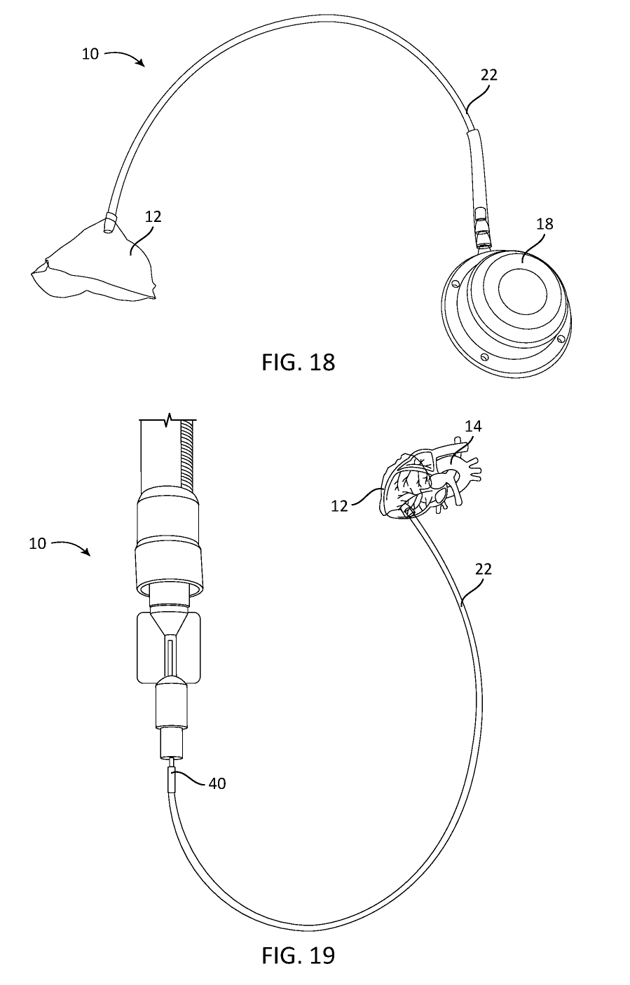

Use of the refillable thericardium 10 in a rat model is illustrated in FIGS. 18-20. A rat-implantation model of a thericardium 10 is shown in FIG. 18. The thericardium 10 includes a subcutaneous port 18 coupled via a catheter 22 with a thericardium sleeve 12 for the rat's heart 14, wherein the thericardium sleeve 12 was shaped to the rat's heart 14 by 3-D printing a mold in the same geometry as a rat heart of the same size. An explanted version of a thericardium 10 is shown in FIG. 19, wherein a catheter 22 is attached to a sleeve 12 that includes a biomaterial and that is sutured to the epicardial surface of a heart 14.

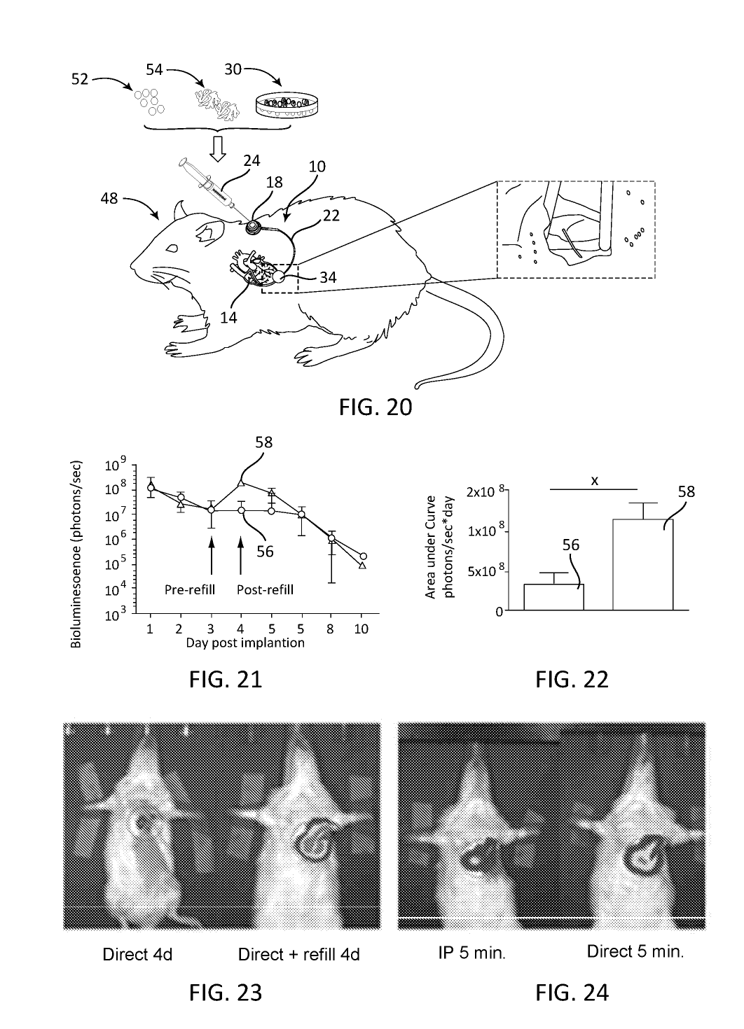

As shown in FIG. 20, the reservoir 34 was implanted on the epicardium of the heart 14 with a catheter 22 leading to a subcutaneous port 18 that allows replenishable, targeted delivery and localized release of drugs 52, macromolecules 54 or cells 30. A macroporous methacrylated gelatin cryogel was used as a biomaterial reservoir 34. The cryogel was implanted directly on the heart 14; the other end of the catheter 22 was connected to a port 18 that is implanted under the skin. For the case of cell delivery, cells were pre-seeded on the reservoir 34, which was then sutured to the epicardium. Therapy can then be seeded onto the biomaterial in situ through the conduit 22.

Refilling the Simplified Thericardium:

a) With Cells

First, we demonstrated the ability to replenish cells in situ via the thericardium 10 (FIGS. 21-23). Luciferase-expressing mMSCs were pre-seeded on to the methacrylated gelatin biomaterial 34; and, at day four, one million cells were replenished onto the biomaterial 34 through the subcutaneous port 18 of the refill group. This refilling (plot 58) resulted in a greater than ten-fold increase in the bioluminescence, as shown in the image to the right in FIG. 23, representing the number of cells at the target site, as compared to non-reloaded gels (plot 56). The areas under the curves of FIG. 21 for each group were calculated after background subtraction are plotted in FIG. 22 and show a significant difference between the “dose” of the cell therapy for the refill group 58.

b) With Small Molecules

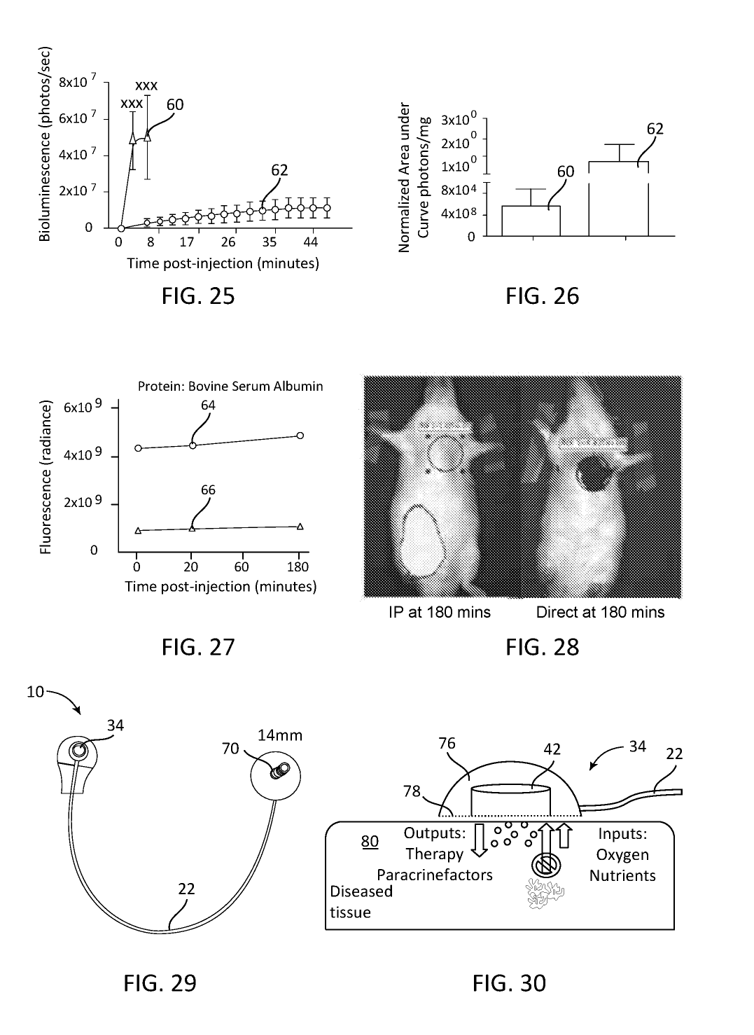

The imaging substrate D-luciferin was used to demonstrate the rapid, targeted delivery of small molecules to the heart 14. The thericardium reservoir 34 was pre-seeded with luciferase expressing mouse mesenchymal cells (mMSCs) before implantation so that bioluminescence of the cells would indicate the presence of imaging substrate. The effect of delivery via the reservoir 34 is an immediate, localized dose to the heart 14, as shown in FIGS. 24-26. Delivery via the port 18 (plot 60) produced a more rapid and intense bioluminescence (as shown in the image to the right in FIG. 24), as compared to intraperitoneal (IP) delivery (plot 62), even though the quantity of delivered substrate via the port 18 was >70-fold less. The net delivery to the target, as indicated by integrating the areas under the curves of FIG. 25 and normalizing by the dose, was dramatically enhanced with delivery via the reservoir system.

c) With Macromolecules

Next, we demonstrated that protein therapeutics could be delivered through the thericardium 10. A bovine serum albumin solution tagged with a fluorescent molecule (Vivotag 800) was delivered via the port 18. After three hours, there was a sustained concentration of the protein at the target site. The same amount of protein was injected intraperitoneally as a control, but an undetectable quantity of the therapy had reached the target site after three hours; the fluorescence signal at the target was equal to background measurements for intraperitoneal delivery, as shown in FIG. 27. The bioluminescence from the dose is shown for intraperitoneal delivery (at left) and for direct delivery via the thericardium 10 is shown in FIG. 28.

Encapsulated Thericardium Device and Refill in a Model of Myocardial Infarction:

Additional control over therapy is enabled by the realization of an encapsulated thericardium to protect therapy from mechanical ejection resulting from a beating heart or potentially the host immune response, and to selectively control the therapy or paracrine factors that pass through a porous membrane onto the diseased tissue. This thericardium is shown in FIG. 29 and is similar to the system previously described with the exception that the reservoir 34 defines an encapsulated space; a biomaterial 42 can be optionally encapsulated in this space. FIG. 30 shows the layers of the reservoir 34; at the tissue 80/membrane 78 interface, the membrane 78 has tuneable porosity and separates the biomaterial 42 from the heart 14, allowing selected therapy to pass, and secures to the catheter 22.

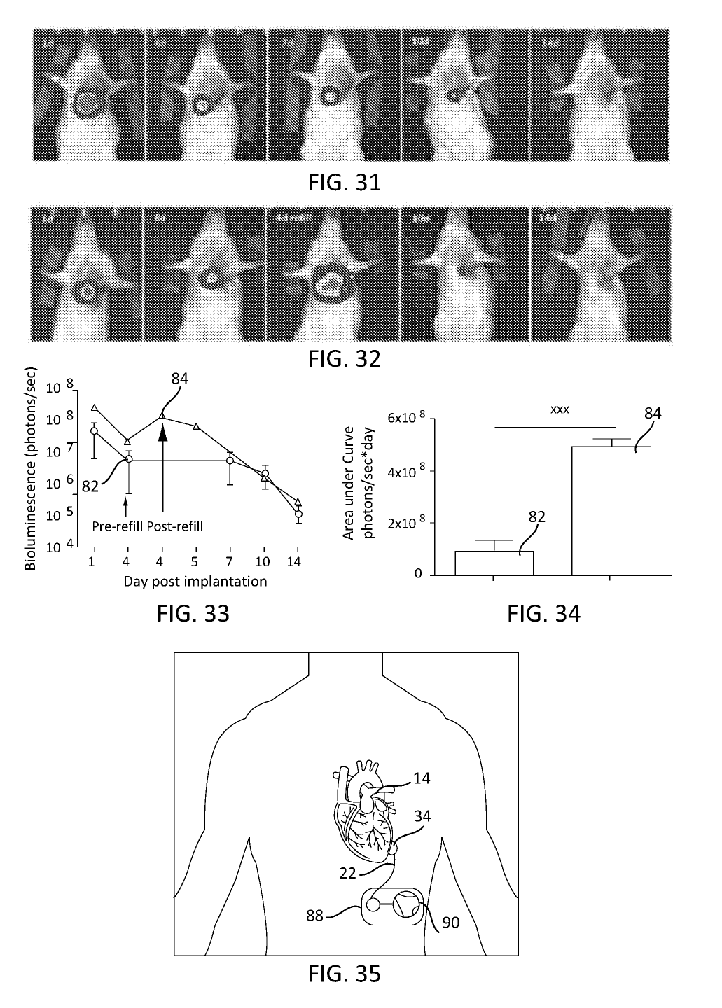

The ability to deliver and replenish cells to a thericardium 10 placed on an infarcted rodent heart 14 was next tested; the study included two groups, direct delivery of a cell-loaded cryogel with the thericardium 10 and the same system with a refill of one million cells at day four post-operatively. Representative bioluminescent images are shown at post-operative time points for a direct delivery group (FIG. 31) and a direct delivery group with refill at day four (FIG. 32). The bioluminescence data (shown in FIGS. 33 and 34) demonstrate that the cell number can be increased by refilling (plot 84) compared with a similar delivery via an encapsulated reservoir 34 without refill (plot 82) and that cell survival at the target site is prolonged in both groups with the encapsulated thericardium, compared to the simplified thericardium with just the biomaterial 42 serving as the reservoir 34, as described previously and as shown in FIG. 20. The areas under the curves 56 and 58 of FIG. 21, which are plotted in FIG. 22, demonstrate that the therapy dose is significantly enhanced with a single refill, and we have demonstrated in-vitro that multiple cell refills are possible with this system.

Design of Reservoir for Encapsulated Therapy Delivery

The membrane immune isolation technology described here can increase transplanted cell retention and survival, enable protection from the host immune response, and can be modified to adjust the type and rate of therapy diffusion from the therapeutic reservoir. This encapsulated delivery technology may be used to enable the isolation and study of the effect of paracrine and autocrine factors produced by transplanted cells for the purposes of cardiac regeneration and to eliminate or reduce the host immune response so as to enable the long-term de novo production and delivery of therapeutic paracrine factors (i.e., operating as a cell factory) from an allogeneic or potentially a xenogeneic cell source, without the need for immunosuppressive regimens. This sustained viability over an extended period of time, within a suitable therapeutic window, could lead to improved clinical outcomes. Additionally, the delivery device may facilitate biopsy in a minimally invasive manner and be ultimately retrievable in the case of unforeseen safety issues.

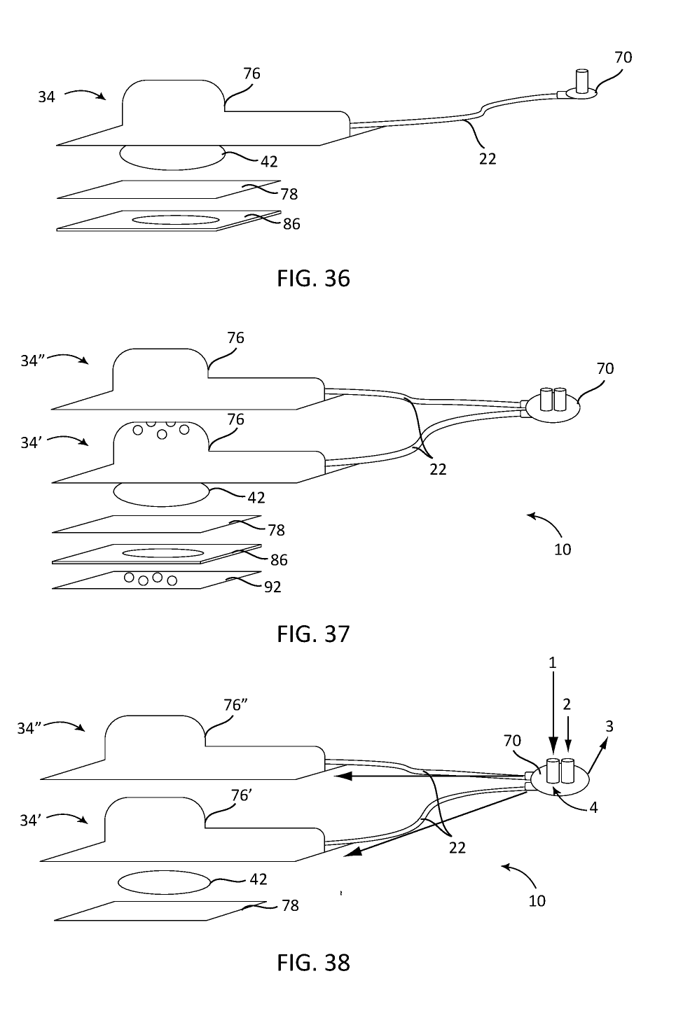

The embodiment of a reservoir 34 shown in the exploded view of FIG. 36 includes an impermeable membrane 76 formed, e.g., of a thermoplastic urethane, in the shape of a pocket. A cryogel biomaterial 42 is contained in the impermeable membrane 76, and a permeable membrane 78 having a modifiable pore size and formed, e.g., of polycarbonate. An opposite side of the pocket is sealed by a thermoplastic urethane layer 86 defining a window through which the polycarbonate membrane 78 is exposed. Therapy passes from the cryogel biomaterial 42 through the permeable membrane 78. The pocket can be refilled via a catheter 22 and a refill port 70. Alternatively, the pocket can be refilled via an implanted supply reservoir 90, as shown in FIG. 35. The pump 88 of FIG. 35 can be used for sustained delivery of exogenous nutrients to a cell biomaterial reservoir 34 and for removal of cell debris.

A thericardium 10 including two reservoir sections 34′ and 34″(with section 34″ stacked on top of section 34′, as shown in FIG. 39) is shown in the exploded view of FIG. 37. The refill port 70 includes two input conduits, each leading to a respective catheter 22 to one of the reservoir sections 34′ and 34″. The first reservoir section 34′ includes an impermeable thermoplastic membrane 76, a cryogel biomaterial 42, a permeable polycarbonate membrane 78, a thermoplastic urethane 86 that defines a window, and a thermoplastic urethane membrane 92.

Another thericardium 10 is shown in the exploded view of FIG. 38. The first reservoir section 34′ in this embodiment defines a cell reservoir pocket containing a shear thinning biomaterial 42 and a tunable-porosity membrane 78. This thericardium 10 can be used to remove and replace the toxic cell/biomaterial microenvironment, which may have suffered mechanical and/or enzymatic degradation. The second reservoir section 34″ serves as an actuation pocket. When using this thericardium 10:
(1) enzymes are target delivered to enzymatically degrade the biomaterial structure 42;
(2) actuation is provided via the application of pressure to the second reservoir section 34″ to deform the first reservoir section 34′ and mechanically disrupt the biomaterial structure 42 in the pocket of the first reservoir section 34′; negative pressure is applied to the pocket to prevent ejection of disrupted biomaterial 42 through the permeable membrane 78;
(3) the degraded solution in the pocket of the first reservoir section 34′ is aspirated from the pocket using the left-most input of port 70; the aspiration-induced flow will lower the viscosity of the remaining shear thinning biomaterial 42; and
(4) new cell-laden biomaterial is injected into the pocket of the first reservoir section 34′,
   wherein reference number (1)-(4), above, correspond to the reference numbers for inputs and outputs at the dual port 70 in FIG. 38.

The biomaterial 42 in this embodiment comprises hyaluronic acid, formed with a 1:1 ratio of pre-polymer to crosslinker, enabling easier injection of accurate volumes, and the degradation enzyme is hyaluronidase. After evacuation of the biomaterial 42 from the reservoir via the above-described procedure, additional biomaterial 42 is refilled via external catheter to avoid blocking the catheter 22 leading to the first reservoir 34′.

In another embodiment, the thericardium 10 of FIG. 38 can be used to remove and replace a toxic cell/biomaterial microenvironment in the pocket of the first reservoir 34′ using a thermoresponsive biomaterial 42 in the first reservoir 34′ and using the second reservoir 34″ as a cooling pocket. In this exemplification:
a) a temperature-changing substance (e.g., a thermoresponsive hydrogel, such as an N-isopropylacrylamide polymer, a poly(ethylene oxide)-b-poly(propylene oxide)-b-poly(ethylene oxide) polymer or a poly(ethylene glycol)-biodegradable polyester copolymer, that utilizes a temperature change as a trigger that determines its gelling behavior) is target delivered via an input (2) through the right-most input of port 70 to the second reservoir 34″ to achieve reverse gelation of a temperature-sensitive biomaterial 42 in the first reservoir 34′;
b) the cooling pocket of the second reservoir 34″ is pressurized via a pressure input (2) that the right-most input of port 70 to mechanically disrupt the biomaterial structure in the cell pocket of the first reservoir 34″; as in the preceding embodiment, negative pressure is applied to the pocket to prevent ejection of disrupted biomaterial 42 through the permeable membrane 78;
c) the degraded solution in the pocket of the first reservoir 34′ is aspirated from the pocket using the left-most input of port 70; the aspiration-induced flow will lower the viscosity of the remaining shear thinning biomaterial 42; and
d) new cell-laden biomaterial is injected into the pocket of the first reservoir 34′,

In additional embodiments, the implanted catheter 22 to the first reservoir 34″ can be used as a guide for a steerable catheter with a suction tip that can be used to the remove the degraded biomaterial 42. The tip can also be cooled or heated to reverse gelate a thermoresponsive hydrogel in the first reservoir 34′. Light, magnetism, an ultrasound frequency emission, or a radiofrequency emission is generated or transmitted from the cather tip to gelate a functionalized biomaterial 42 for delivery or to degrade it for evacuation.

### DISCUSSION

The thericardium 10 can function as a delivery system that allows targeted, replenishable and sustained presentation of cellular and molecular therapy to the heart 14. A biomaterial-based reservoir (gelatin cryogel) 34 initially seeded with luciferase-expressing mouse mesenchymal stem cells, was attached to the epicardial surface of the infarcted rat heart 14. Gelatin is derived from collagen and contains inherent peptide sequences that facilitate cell adhesion and enzymatic degradation. Due to its low cost, lack of immunogenicity, and previous use in medicine as a hemostatic agent and blood volume expander, gelatin is an attractive implantable biomaterial. However, we foresee that this platform can be extended to other biomaterials that have demonstrated an ability to increase cell retention at the heart (e.g., alginate, chitosan, hyaluronic acid-based gels, gelfoam, and collagen).

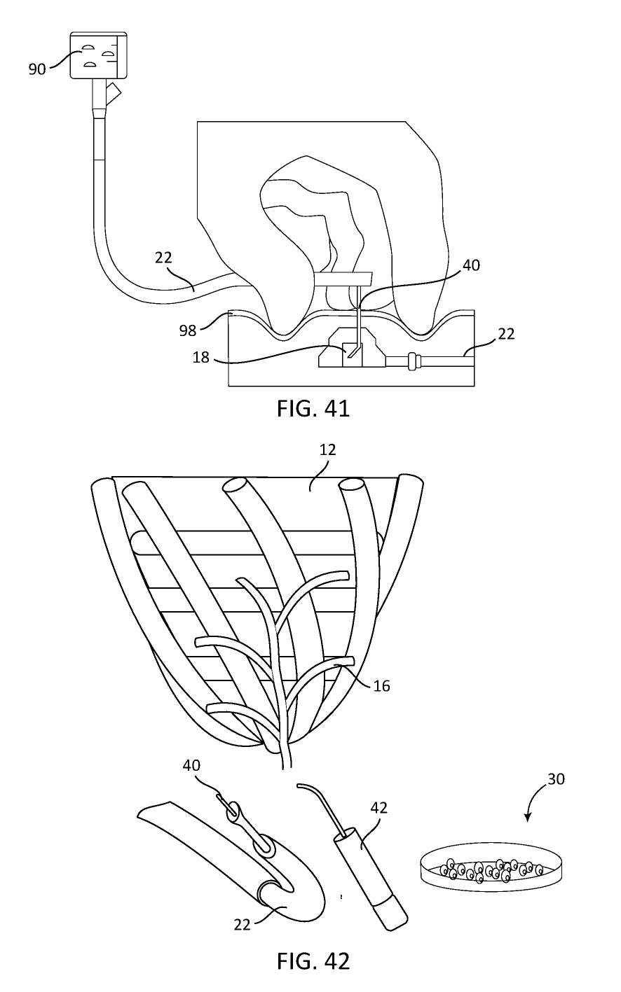

An implantable catheter 22 was used as a conduit between this reservoir 34 and a subcutaneous port 18 located at a dorsal site of the rat. The biomaterial reservoir 34 can be refilled with cells through the port 18 at defined points in time, increasing the resident cell number 10-fold. Although just one refill was conducted in vivo, the possibility for multiple refills and replenishments with similar or different therapies exists and was demonstrated in vitro. Furthermore, attaching the catheter 22 to a small implanted, refillable pump 88 (for example, a stem cell pump from BioLeonhardt of Santa Monica, Calif., USA) enables a sustained infusion (as depicted in FIG. 35). The ability of the system to allow targeted injection of molecular therapies directly to the biomaterial reservoir 34 in contact with the heart 14 was demonstrated by delivering bovine serum albumin and the imaging substrate D-Luciferin from a remote therapy supply 90 through the subcutaneous port 18 implanted under the skin 98 (as shown in FIGS. 40 and 41), both indicating a rapid, localized delivery of therapy, which can improve efficiency of drugs and reduce off-target adverse effects.

By enabling triggered, localized release of treatment, the thericardium 10 can “deliver the right treatment at the right time” to the patient. The pericardium is a fluid filled sac that forms a natural barrier surrounding the heart 14. Targeting drugs directly to the heart by delivering to the pericardial space [i.e., intrapericardial (IPC) delivery] can serve as an advantageous strategy to obtain higher drug efficiencies, while lessening the side effects. Oral formulations are the most commonly used and most patient-acceptable method of drug delivery; oral formulations, however, have many inherent limitations including incomplete absorption through the gastrointestinal mucosa, poor bioavailability and poor compliance. Intravenous (IV) administration overcomes these issues by bypassing absorption and first-pass metabolism. For both oral and IV delivery, however, inter-patient pharmacokinetic variability can cause extensive deviations in the amount of drug that reaches the desired molecular target; and significant quantities of drug reach off target sites, potentially causing side effects. This off-target delivery is a particularly important problem for drugs with a narrow therapeutic index, as a high concentration can cause toxic side effects while a low concentration can eliminate any clinical benefit.

Localized delivery confers the advantages of greater control over desired tissue exposure, decreased variability of clinical response, lower needed therapeutic doses, and opportunities to use bioagents with a short half-life or that are biologically incompatible with the gastro-intestinal tract and blood stream (e.g., cells and their secreted paracrine factors). The efficacy of IPC drug delivery to the heart has been studied for angiogenic substances and vasodilators as well as rhythm management drugs (anti-arrhythmics, arrhythmic agents. Hermans, et al., in “Pharmacokinetic advantage of intrapericardially applied substances in the rat”, 301 J. Pharmacol. Exp. Ther. 672-678 (2002), used a chronic administration animal model to show pharmacokinetic advantages in the rat with IPC infusion. Van Brakel et al, showed that this technique improved the efficacy of β-blockers sotalol and atenolol compared to IV administration in “Intrapericardial delivery enhances cardiac effects of sotalol and atenolol”, 44 J. Cardiovasc. Pharmacol. 50-56 (2004). However, easy and reproducible access has been a major limiting factor for IPC delivery. The direct, refillable thericardium demonstrated herein suggests that clinical translation of IPC drug delivery may be readily obtained. In a broader sense, this system provides a platform for delivery to other diseased tissues, as well, for other therapeutic regimens with a narrow therapeutic index.

In terms of clinical translation, the thericardium 10 and the disclosed methods of implantation and functional monitoring have a potential to prove extremely beneficial for enabling sustained delivery of the paracrine factors released from transplanted cells in close proximity to the diseased tissue of the heart 14 and for allowing further research studies of the effect of multiple administrations of cells that have previously been infeasible due to the prohibitive nature of multiple invasive surgeries. Furthermore, the thericardium 10 enables the multimodal localized delivery of different molecular therapies (e.g., cells, small molecules, and macromolecules) in an attempt to mimic or modify the inherently complex physiological and pathological processes in the heart 14. In addition to temporal control, multiple reservoirs 34 enable spatial control and multimodal treatment regimens to different parts of the heart 14; for example, delivery of pro-regenerative therapy to the left ventricle and anti-arrhythmic therapy to the left atrium. Finally, the reservoir(s) 34 may be implanted without therapy and filled with therapeutic cargo non-invasively after a certain amount of time. This procedure is advantageous for previously reported work with autologous stem cells, when, for example, a biopsy can be taken at the time of implantation of the thericardium 10; then, stem cells can be cultured and expanded and re-implanted through the thericardium 10 after a number of weeks or months without the need for an additional surgery. An encapsulated thericardium 10 may potentially enable the long-term de novo production and delivery of therapeutic paracrine factors from a transplanted cell source without the need for immunosuppressive regimens.

To maximize the potency of cell therapy, systems that can longitudinally monitor the viability and function of transplanted cells in vivo would be beneficial. The thericardium 10, described herein, can address this need by having additional utility as an enhanced imaging method for quantifying cell number on the heart 14. In this case, luciferase-expressing cells are used, and D-luciferin can be injected directly through the thericardium 10, requiring much less substrate and reducing the duration of time that the animal is under anesthesia. This capacity for enhanced imaging represents a considerable advantage in terms of convenience, cost, consistency and time taken to conduct animal imaging. It can allow imaging in less than five minutes with 50 μl/0.75 mg of D-luciferin, compared to IP injection that can require more than 45 minutes for D-luciferin circulation, and up to 3.5 ml/52.5 mg of D-luciferin, thereby facilitating more frequent imaging and a more accurate pharmacokinetic profile. This system can also be used, if desired, for injection of media or nutrients into the reservoir 34 to prolong cell survival. Biosensors can also be injected and retrieved locally to monitor biomarkers indicative of disease. With the rate-limiting membrane 78 surrounding the reservoir 34, microneedle technology can be used to allow direct injection into tissue. The potential of the system for monitoring and feedback is vast.

Finally, pressure-volume loop analysis has become the “gold standard” for measuring hemodynamic parameters in research models. Additionally, lessons from clinical trials show us that, although the ejection fraction (usually determined by echocardiography or magnetic resonance imaging) has been regarded as the gold standard for assessing outcomes, it may not be the most suitable for assessing the effects of cell therapy-pressure-volume loop analysis allows recording of multiple hemodynamic parameters that can be used for this purpose. Previous studies using a pressure-volume catheter, and the apical stick method terminated the experiment after measurements were conducted.

Here, we demonstrate a survival study that allows repeated measurements on the same animals, enabling a longitudinal study on an animal following the progression of post-myocardial-infarction necrosis, scarring and remodeling. The ability to follow disease progression and relate it to cell dose and viability, afforded by the thericardium 10, is a considerable advantage for assessing pre-clinical treatments and can potentially help to avoid the unpredictable efficacy of regenerative therapies when implemented in clinical trials. In addition to these advantages, this capacity allows the use of fewer animals for experimental groups. Although this approach has been reported using the carotid access methods previously but not, to the inventors' knowledge, for the apical stick (or so-called “open-chest” method), which is a much more straightforward procedure. A potential challenge with a repeated measurement technique in the carotid artery is thrombosis, where mechanical movement and scraping of the catheter can lead to endothelium damage, so coagulation is advantageously monitored. Advantages of the repeated apical stick method, as compared to carotid access, are that proper placement is easier to confirm, and carotid placement may be prohibited if the carotid is atherosclerotic (e.g., as in ApoE mice) or the aortic valve is calcified (e.g., hypertrophy and heart failure models).

We can draw the following six conclusions from this study: (i) implantation of the thericardium 10 on the heart 14 with a conduit 22 connecting the reservoir 34 to a subcutaneous access port 18 is possible in a rat model; (ii) the system enables non-invasive replenishment of cells to the thericardium reservoir 34 and improves cell number at the site; (iii) the system can be refined with another rate-limiting layer to enhance therapy selectivity; (iv) the technology also allows rapid, targeted delivery of macromolecules and small molecules directly to the site; (v) the implanted system constitutes a rapid, inexpensive and safe method for bioluminescent quantification of cell number by direct administration of an imaging substrate during in vivo imaging; and (vi) a method for longitudinal hemodynamic measurements using a pressure-volume catheter with the apical stick method can be used to quantify cardiac function in a survival animal model.

### Additional Embodiments

Inventive concepts described herein can also be incorporated into a variety of other embodiments, including the following.
a) Biomaterial liner: As shown in FIGS. 7 and 8, a liner 19 is fabricated from a biomaterial [e.g., alginate, poly-lactic-co-glycolic acid (PLGA), poly-l-lactide acid (PLLA), gelatin cryogel, or alginate polyacrylamide] to conform to the 3D surface of the heart. The liner 19 can be produced, e.g., via casting, 3D printing, molding, laser cutting, or bonding. The liner 19 can be formed with encapsulated cells (e.g., in discrete reservoirs 34) or can be seeded with cells (where the entire liner 19 serves as a reservoir 34). Cells, growth factors or therapy can be refilled through inbuilt channels (conduits) 16 in the device leading directly to the liner 19.
b) Injection: As shown in FIG. 42, cells 30 can be myocardially injected through the cardiac sleeve 12. Cells 30 are initially injected into the myocardium. Further injections are enabled via catheter delivery (e.g., using a curved catheter 22 with a retractable needle 40) through channels (conduits) 16. In additional embodiments, a fully implantable system is used, wherein delivery of cells 30 (or other therapy) is achieved by implanting a supply reservoir 90, e.g., in the body in an area remote from (or proximate to) the heart 14 and coupled with the reservoir(s) 34 in contact with the heart 14 via a conduit, as shown in FIG. 35; and the cells 30 or other therapy is pumped (via infusion pump 88) or otherwise delivered from the supply reservoir 90 (e.g., via a stimulus external to the body) at controlled intervals to the sleeve 12 to refill the reservoir(s) 34. In a particular embodiment, the supply reservoir 90 is incorporated in a microchip drug delivery device (available from Microchips Biotech, Inc., of Lexington, Mass.) or a similar device that can achieve remote/external triggered drug release.
c) Combination mechanical and biological therapy: By combining the thericardium 10 with a cardiac assist device (e.g., a cardiac compression device 32), a combination mechanical (e.g., assisting with the pumping of the heart 14) and biological therapeutic strategy can be implemented, as shown in FIG. 17 and as described in published PCT Application No. WO 2015051380 A2. Advantages of this polytherapeutic approach are that it addresses both the short and long-term needs of the heart 14 for mechanical assistance and myocardial regeneration.
d) Customization of sleeve based on clinical (MRI/CT) patient data: The thericardium sleeve 12 can be manufactured by reconstructing clinical data pertaining to a patient-specific heart 14 to a 3D computer model. For example, this reconstruction can be performed by forming a mold, wherein the casting surface of the mold mimics the surface of the patient's heart 14. The thericardium sleeve 12 can be cast in silicone, urethane or any other flexible material on the mold and can thereby be shaped to conform closely to the particular heart 14 with which it will be brought into contact to provide therapy. Other rapid prototyping methods can alternatively be used for the reconstruction.
e) 3D printing of sleeve: The entire sleeve 12 (including reservoirs and channels) or part(s) thereof can be formed by 3D printing a flexible material or a biomaterial to produce the desired sleeve shape. Again, the sleeve 12 can be printed by reconstructing clinical data pertaining to a patient-specific heart to a 3D computer model and printing the flexible material or biomaterial in conformance with the 3D computer model's surface.
f) Responsive delivery: Cells, growth factors, conditioned media (i.e., media conditioned with cultured cells), and/or small molecules that help regulate a biological process can be delivered through the thericardium 10; or drugs in response to an in vivo physiological change—e.g., change in the rate/rhythm of the heart during atrial fibrillation (mechanical) or release of troponins (biomarkers) during a myocardial infarction can be delivered through the thericardium 10. Mechanical massage of the tissue can help therapy and also, itself, help with regeneration.
g) Electrode: An electrode can be inserted through at least one of the ports 18/70 and through catheters 22 for localized directed electrical cardioversion of the heart 14 to treat a cardiac arrhythmia.

### Additional Applications:

Beyond the heart therapy applications, described above, the methods and apparatus described herein can be used in a variety of other applications, such as the following:
a) maintenance of donor organs for transplanting, e.g., by wrapping the removed organ in a sleeve 12 during ex vivo storage and transport and delivering sustaining therapy (e.g., electrolytes and salts to maintain the organ or anti-rejection drugs) to the organ through the reservoirs 34;
b) delivery of anti-rejection therapy to the heart 14 or to other transplanted organs by implanting a sleeve 12 around the organ after the transplant is completed and delivering therapy to the transplanted organ there through;
c) delivery of anti-arrhythmic drugs to a heart 14 through a thericardium 10 in which the heart 14 is contained;
d) on-demand therapy delivery based on an external stimulus (e.g., magnet, ultrasound, or mechanical stimulation from a second pocket in the thericardium);
e) temporal control over drug release using reservoirs 34 with different porositiess (e.g., using smaller pore sizes or lower pore density in the layer covering the reservoir(s) 34 to release a drug contained in the reservoir(s) more slowly); using different biomaterials with different compositions, porosities, and/or biodegradation rates; or using an infusion pump;
f) delivery of cardioprotective drugs to the heart 14;
g) local delivery of cell or molecular therapy (e.g., T cells expressing chimeric antigen receptors) to other organs (for example, to the stomach, pancreas, or liver);
h) prevention of adhesions (synthetic pericardium) to a transplanted heart 14 and prevention of adhesions (synthetic pericardium) when implanting left ventricular assist device or artificial heart, allowing removal of the assist device or artificial heart;
i) external transdermal use against the skin for wound healing, diabetic neuropathies, treating gangrenous or necrotic ulcers, etc.;
j) transdermal delivery of hormones (e.g., hormone replacement therapy or T3 for thyroid function);
k) localized delivery of dopamine into the brain or delivery of levodopa close to the blood/brain barrier for Parkinsons patients;
l) treating gastro-intestinal conditions [e.g., triple therapy for helicobacter pylori and gastric ulcers (2 antibiotics plus proton pump inhibitor), where the methods described herein would allow localized delivery that could decrease antibiotic resistance and overcome the patient compliance issue or localized delivery of anti-inflammatory drugs, or delivering immunosuppressants for Crohn's disease or ulcerative colitis;
m) localized delivery of anti-thyroid pharmacotherapy for the treatment of hyperthyroidism;
n) implantation of an intrauterine biodegradable antimicrobial eluting apparatus for the treatment of chronic/acute uterine tract infections;
o) localized delivery of lidocaine for the treatment of chronic pain at a pain site;
p) localized delivery of antibiotics to prevent/treat infection in artificial joints or to treat osteomyelitis; and
q) delivery of therapy for diabetes.

Further examples consistent with the present teachings are set out in the following numbered clauses:
1. A method for providing therapy to living tissue, comprising:
   contacting living tissue with at least one reservoir loaded with cells or a therapeutic composition, wherein the reservoir is in fluid communication with at least one conduit that includes a refilling port;
   releasing a constituent selected from (a) cells, (b) bioagents from the cells or (c) the therapeutic composition from the reservoir to the living tissue;
   refilling the reservoir with (i) cells, (ii) nutrients for cells, or (iii) additional therapeutic composition; and
   continuing to release (a) cells, (b) bioagents from the cells or (c) the therapeutic composition from the reservoir to the living tissue after the refilling.
2. The method of clause 1, wherein the reservoir is implanted in a living organism when the reservoir contacts the living tissue.
3. The method of any of the preceding clauses, wherein the living tissue is heart tissue.
4. The method of clause 3, wherein the reservoir is incorporated in a thericardium that replaces or is placed inside a pericardium to contain the heart inside an organism.
5. The method of clause 3 or 4, further comprising assisting pumping of the heart or directly stimulating the heart with the thericardium.
6. The method of any of the preceding clauses, wherein the tissue forms an organ, and wherein the reservoir is incorporated in a sleeve that contains the organ outside an organism from which it was extracted during a transplant procedure.
7 The method of any of the preceding clauses, wherein a plurality of reservoirs are contacted with the living tissue and refilled.
8. The method of any of the preceding clauses, wherein cells, from which the bioagent is released, are retained on or in the reservoir.
9. The method of clause 8, wherein the cells are adhered to a biomaterial cell carrier inside the reservoir.
10. The method of clause 9, wherein the biomaterial cell carrier is selected from a porous biomaterial in the form of a cryogel, hydrogel, or scaffold, a methacrylated gelatin, alginate, collagen, chitosan, poly-lactic-co-glycolic acid (PLGA), poly-l-lactide acid (PLLA), extracellular matrix, fibrin, alginate polyacrylamide, and hyaluronic acid.
11. The method of clause 8, wherein the reservoir is a biomaterial cell carrier.
12. The method of any of clauses 8-11, further comprising providing dynamic mechanical actuation to the cells.
13. The method of any of clauses 8-12, further comprising using a pump to pump nutrients to the cells and remove cell debris from the reservoir.
14. The method of any of the preceding clauses, wherein the reservoir comprises a permeable membrane through which the constituent is released, wherein the permeable membrane contacts the living tissue.
15. The method of any of the preceding clauses, wherein the reservoir is configured with an actuator that forces the constituent from the reservoir.
16. The method of any of the preceding clauses, wherein the reservoir comprises a porous scaffold that contacts the living tissue.
17. The method of any of the preceding clauses, wherein the reservoir is incorporated in a sleeve.
18. The method of clause 17, wherein the tissue forms an organ, the method further comprising:
   imaging the organ;
   forming a 3D model of a surface of the organ from the images; and
   fabricating the sleeve with a shape that conforms to the surface of the imaged organ.
19. The method of clause 18, wherein the sleeve is fabricated via 3D printing of a mold, and forming or casting the sleeve with the mold.
20. The method of any of clauses 17-19, further comprising inserting the sleeve into a living organism and into contact the living tissue, wherein the sleeve gradually biodegrades to eventually eliminate the sleeve inside the living organism.
21. The method of any of clauses 17-20, further comprising attaching the sleeve to the tissue or to an adjoining structure in a living organism.
22. The method of any of the preceding clauses, wherein the reservoir contains a therapeutic composition selected from chemotherapeutic agents, immunosuppressants, culture media, conditioned media, small molecules that help regulate a biological process, anti-rejection drugs, anti-arrythmic drugs, anti-anginal drugs, cardioprotective drugs, hormones, dopamine, levodopa, antibiotics, anti-inflammatory drugs, anti-thyroid pharmacotherapy, anti-microbial drugs, and lidocaine.
23. The method of any of the preceding clauses, further comprising passing at least one catheter through the conduit(s).
24. The method of clause 23, further comprising using the catheter to adjust the position of the reservoir along the tissue inside a living organism.
25. The method of clause 23, further comprising using the catheter to anchor the reservoir to the tissue inside a living organism.
26. The method of clause 23, further comprising using the catheter to configure the reservoir with an actuator, wherein the actuator adjusts the position of the reservoir along the tissue inside the living organism.
27. The method of any of the preceding clauses, further comprising releasing the constituent from the reservoir over a temporally controlled schedule.
28. The method of any of the preceding clauses, wherein the reservoir includes microneedles that inject the bioagents or the therapeutic composition directly into the living tissue.
29. The method of any of the preceding clauses, wherein the reservoir includes at least two sections defining respective pockets, wherein a first pocket of a first section contains the cells or the therapeutic composition, and wherein a second pocket of a second section functions as an actuator.
30. The method of clause 29, further comprising changing the pressure in the second pocket to actuate release of the constituent from the first pocket.
31. The method of clause 29, wherein the first pocket contains a thermoresponsive biomaterial, the method further comprising generating a temperature change in the second pocket to actuate the thermoresponsive biomaterial.
32. The method of any of the preceding clauses, further comprising releasing a luminescent agent from the reservoir.
33. A tissue therapy apparatus comprising:
   at least one reservoir including a porous wall through which contents of the reservoir can pass;
   a conduit including a first end and a second end, wherein the second end is in fluid communication with the reservoir; and
   a refill port mounted at the second end of the conduit.
34. The tissue therapy apparatus of clause 33, further comprising a sleeve in which the reservoir is incorporated.
35. The tissue therapy apparatus of clause 34, wherein the sleeve includes micro-patterning or an adhesive material to adhere the sleeve to tissue that is to receive therapy from the reservoir.
36. The tissue therapy apparatus of any of the preceding clauses, including a plurality of the reservoirs in fluid communication with the conduit or with one or more additional conduits with refill ports at their second ends.
37. The tissue therapy apparatus of clause 36, wherein at least one of the reservoirs contains contents distinct from contents contained in other reservoirs.
38. The tissue therapy apparatus of clause 36, wherein, to enable differentiated release of contents from different reservoirs:
   (a) the porous wall of at least one of the reservoirs has a porosity different from the porous wall of another of the reservoirs; or
   (b) at least one of the reservoirs has a thickness that is different from another of the reservoirs.
39. The tissue therapy apparatus of any of the preceding clauses, wherein the reservoir contains a therapeutic composition.
40. The tissue therapy apparatus of any of the preceding clauses, wherein the reservoir contains cells.
41. The tissue therapy apparatus of clause 40, wherein the cells are adhered to a biomaterial cell carrier inside the reservoir.
42. The tissue therapy apparatus of clause 41, wherein the biomaterial cell carrier is selected from a porous cryogel, a methacrylated gelatin, alginate, collagen, chitosan, poly-lactic-co-glycolic acid (PLGA), poly-l-lactide acid (PLLA), and alginate polyacrylamide.
43. The tissue therapy apparatus of any of the preceding clauses, wherein the reservoir contains a therapeutic composition selected from a chemotherapeutic agents, immunosuppressants, cultured media, small molecules that help regulate a biological process, anti-rejection drugs, anti-arrythmic drugs, and cardioprotective drugs.
44. The tissue therapy apparatus of any of the preceding clauses, further comprising an actuator configured to pump contents of the reservoir from the reservoir.
45. The tissue therapy apparatus of any of the preceding clauses, wherein the porous wall of the reservoir comprises a urethane.
46. A method for providing therapy to living tissue, comprising:
   contacting living tissue with a sleeve through which conduits pass, wherein the conduits each include a first open end in fluid communication with the living tissue, with a biomaterial on the tissue, or with a reservoir containing the biomaterial and including a porous membrane at an interface of the reservoir with the tissue; and
   periodically injecting at least one of (a) cells, (b) bioagents from the cells and (c) a therapeutic composition through the conduits into contact with the living tissue.
47. The method of clause 46, further comprising inserting a catheter through at least one of the conduits, wherein the injection is performed via the catheter.

In describing embodiments of the invention, specific terminology is used for the sake of clarity. For the purpose of description, specific terms are intended to at least include technical and functional equivalents that operate in a similar manner to accomplish a similar result. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties or other values are specified herein for embodiments of the invention, those parameters or values can be adjusted up or down by 1/100^{th}, 1/50^{th}, 1/20^{th}, 1/10^{th}, ⅕^{th}, ⅓^{rd}, ½, ⅔^{rd}, ¾^{th}, ⅘^{th}, 9/10^{th}, 19/20^{th}, 49/50^{th}, 99/100^{th}, etc. (or up by a factor of 1, 2, 3, 4, 5, 6, 8, 10, 20, 50, 100, etc.), or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention. Further still, other aspects, functions and advantages are also within the scope of the invention; and all embodiments of the invention need not necessarily achieve all of the advantages or possess all of the characteristics described above. Additionally, steps, elements and features discussed herein in connection with one embodiment can likewise be used in conjunction with other embodiments. The contents of references, including reference texts, journal articles, patents, patent applications, etc., cited herein are hereby incorporated by reference in their entirety; and appropriate components, steps, and characterizations from these references may or may not be included in embodiments of this invention. Still further, the components and steps identified in the Background section are integral to this disclosure and can be used in conjunction with or substituted for components and steps described elsewhere in the disclosure within the scope of the invention. In method claims, where stages are recited in a particular order—with or without sequenced prefacing characters added for ease of reference—the stages are not to be interpreted as being temporally limited to the order in which they are recited unless otherwise specified or implied by the terms and phrasing.

What is claimed is:

1. A method for providing therapy to living tissue, comprising:
contacting living tissue with at least one reservoir that contains cells or a therapeutic composition, wherein the reservoir is in fluid communication with at least one conduit that includes a refilling port;
releasing a constituent selected from (a) cells, (b) bioagents from the cells or (c) the therapeutic composition from the reservoir to the living tissue;
refilling the reservoir with (i) cells, (ii) nutrients for cells, or (iii) additional therapeutic composition; and
continuing to release (a) cells, (b) bioagents from the cells or (c) the therapeutic composition from the reservoir to the living tissue after the refilling.

2. The method of claim 1, wherein the reservoir is implanted in a living organism when the reservoir contacts the living tissue.

3. The method of claim 2, wherein the living tissue is heart tissue.

4. The method of claim 3, wherein the reservoir is incorporated in a thericardium that replaces or is placed inside a pericardium on the heart inside an organism.

5. The method of claim 4, further comprising assisting pumping of the heart or directly stimulating the heart with the thericardium.

6. The method of claim 1, wherein the living tissue forms an organ, and wherein the reservoir is incorporated in a sleeve that contains the organ outside an organism from which it was extracted during a transplant procedure.

7. The method of claim 1, wherein a plurality of reservoirs are contacted with the living tissue and refilled.

8. The method of claim 1, wherein cells, from which the bioagent is released, are retained on or in the reservoir.

9. The method of claim 8, wherein the cells are adhered to a biomaterial cell carrier inside the reservoir.

10. The method of claim 9, wherein the biomaterial cell carrier is selected from a porous biomaterial in the form of a cryogel, hydrogel, or scaffold, a methacrylated gelatin, alginate, collagen, chitosan, poly-lactic-co-glycolic acid (PLGA), poly-l-lactide acid (PLLA), extracellular matrix, fibrin, alginate polyacrylamide, and hyaluronic acid.

11. The method of claim 8, wherein the reservoir is a biomaterial cell carrier.

12. The method of claim 8, further comprising providing dynamic mechanical actuation to the cells.

13. The method of claim 8, further comprising using a pump to pump nutrients to the cells and remove cell debris from the reservoir.

14. The method of claim 8, wherein the reservoir comprises a permeable membrane through which the constituent is released, wherein the permeable membrane contacts the living tissue.

15. The method of claim 1, wherein the reservoir is configured with an actuator that forces the constituent from the reservoir.

16. The method of claim 1, wherein the reservoir comprises a porous scaffold that contacts the living tissue.

17. The method of claim 1, wherein the reservoir is incorporated in a sleeve.

18. The method of claim 17, wherein the tissue forms an organ, the method further comprising:
imaging the organ;
forming a 3D model of a surface of the organ from the images; and
fabricating the sleeve with a shape that conforms to the surface of the imaged organ.

19. The method of claim 18, wherein the sleeve is fabricated via 3D printing of a mold, and forming or casting the sleeve with the mold.

20. The method of claim 17, further comprising inserting the sleeve into a living organism and into contact the living tissue, wherein the sleeve gradually biodegrades to eventually eliminate the sleeve inside the living organism.

21. The method of claim 17, further comprising attaching the sleeve to the tissue or to an adjoining structure in a living organism.

22. The method of claim 1, wherein the reservoir contains a therapeutic composition selected from chemotherapeutic agents, immunosuppressants, culture media, conditioned media, small molecules that help regulate a biological process, anti-rejection drugs, anti-arrythmic drugs, anti-anginal drugs, cardioprotective drugs, hormones, dopamine, levodopa, antibiotics, anti-inflammatory drugs, anti-thyroid pharmacotherapy, anti-microbial drugs, and lidocaine.

23. The method of claim 1, further comprising passing at least one catheter through the conduit(s).

24. The method of claim 23, further comprising using the catheter to adjust the position of the reservoir along the tissue inside a living organism.

25. The method of claim 23, further comprising using the catheter to anchor the reservoir to the tissue inside a living organism.

26. The method of claim 23, further comprising using the catheter to configure the reservoir with an actuator, wherein the actuator adjusts the position of the reservoir along the tissue inside a living organism.

27. The method of claim 1, further comprising releasing the constituent from the reservoir over a temporally controlled schedule.

28. The method of claim 1, wherein the reservoir includes microneedles that inject the bioagents or the therapeutic composition directly into the living tissue.

29. The method of claim 1, wherein the reservoir includes at least two sections defining respective pockets, wherein a first pocket of a first section contains the cells or the therapeutic composition, and wherein a second pocket of a second section functions as an actuator.

30. The method of claim 29, further comprising changing the pressure in the second pocket to actuate release of the constituent from the first pocket.

31. The method of claim 29, wherein the first pocket contains a thermoresponsive biomaterial, the method further comprising generating a temperature change in the second pocket to actuate the thermoresponsive biomaterial.

32. The method of claim 1, further comprising releasing a luminescent agent from the reservoir.

33. A tissue therapy apparatus comprising:
at least one reservoir including a porous wall through which contents of the reservoir can pass;
a conduit including a first end and a second end, wherein the second end is in fluid communication with the reservoir; and
a refill port mounted at the second end of the conduit.

34. The tissue therapy apparatus of claim 33, further comprising a sleeve in which the reservoir is incorporated.

35. The tissue therapy apparatus of claim 34, wherein the sleeve includes micro-patterning or an adhesive material to adhere the sleeve to tissue that is to receive therapy from the reservoir.

36. The tissue therapy apparatus of claim 33, including a plurality of the reservoirs in fluid communication with the conduit or with one or more additional conduits with refill ports at their second ends.

37. The tissue therapy apparatus of claim 36, wherein at least one of the reservoirs contains contents distinct from contents contained in other reservoirs.

38. The tissue therapy apparatus of claim 36, wherein, to enable differentiated release of contents from different reservoirs:
(a) the porous wall of at least one of the reservoirs has a porosity different from the porous wall of another of the reservoirs; or
(b) at least one of the reservoirs has a thickness that is different from another of the reservoirs.

39. The tissue therapy apparatus of claim 33, wherein the reservoir contains a therapeutic composition.

40. The tissue therapy apparatus of claim 33, wherein the reservoir contains cells.

41. The tissue therapy apparatus of claim 40, wherein the cells are adhered to a biomaterial cell carrier inside the reservoir.

42. The tissue therapy apparatus of claim 41, wherein the biomaterial cell carrier is selected from a porous cryogel, a methacrylated gelatin, alginate, collagen, chitosan, poly-lactic-co-glycolic acid (PLGA), poly-l-lactide acid (PLLA), and alginate polyacrylamide.

43. The tissue therapy apparatus of claim 33, wherein the reservoir contains a therapeutic composition selected from a chemotherapeutic agents, immunosuppressants, cultured media, small molecules that help regulate a biological process, anti-rejection drugs, anti-arrythmic drugs, and cardioprotective drugs.

44. The tissue therapy apparatus of claim 33, further comprising an actuator configured to pump contents of the reservoir from the reservoir.

45. The tissue therapy apparatus of claim 33, wherein the porous wall of the reservoir comprises a urethane.

46. A method for providing therapy to living tissue, comprising:
contacting living tissue with a sleeve through which conduits pass, wherein the conduits each include a first open end in fluid communication with the living tissue, with a biomaterial on the tissue, or with a reservoir containing the biomaterial and including a porous membrane at an interface of the reservoir with the tissue; and
periodically injecting at least one of (a) cells, (b) bioagents from the cells and (c) a therapeutic composition through the conduits into contact with the living tissue.

47. The method of claim 46, further comprising inserting a catheter through at least one of the conduits, wherein the injection is performed via the catheter.
